(19) United States
(12) Patent Application Publication
(10) Pub. No.: US 2009/0136598 A1
(43) Pub. Date: May 28, 2009

(54) Compositions for the Treatment and Prevention of Eyelid Swelling

(75) Inventors: Matthew Jonathan Chapin, Amesbury, MA (US); Mark Barry Abelson, Andover, MA (US); Keith Jeffrey Lane, Somerville, MA (US); George Minno, Suwanee, GA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: ACIEX, INC., Westborough, MA (US)

(21) Appl. No.: 12/266,396

(22) Filed: Nov. 6, 2008

Related U.S. Application Data
(63) Continuation-in-part of application No. 11/796,278, filed on Apr. 26, 2007.
(60) Provisional application No. 60/794,983, filed on Apr. 26, 2006.
(60) Provisional application No. 60/845,479, filed on Sep. 18, 2006.
(60) Provisional application No. 61/007,511, filed on Nov. 8, 2007.

(51) Int. Cl. A61K 33/14; A61K 31/047; A61K 31/4164; A61P 27/02

(52) U.S. Cl. 424/680; 514/738; 514/400; 514/396

(57) ABSTRACT

The invention features topical formulations comprising an osmotically active agent and/or a vasoconstrictor and/or an astringent agent for the treatment and prevention of eyelid swelling, and methods of use thereof.

### RELATED APPLICATIONS

This application is a continuation-in-part of U.S. application Ser. No. 11/796,278, filed Apr. 26, 2007, which claims the benefit of U.S. Provisional Application No. 60/794,983, filed Apr. 26, 2006 and U.S. Provisional Application No. 60/845,479, filed Sep. 18, 2006; and this application claims priority to U.S. Provisional Application No. 61/007,511 filed Nov. 8, 2007, the contents of which are each hereby incorporated by reference in their entireties.

### FIELD OF THE INVENTION

The present invention relates to novel ophthalmic compositions and methods useful for the prevention and treatment of eyelid swelling. Specifically, the invention relates to an ophthalmic composition comprising an osmotically active agent, an astringent, a vasoconstrictor, or a combination thereof, useful for the prevention and treatment of eyelid swelling. The invention additionally relates to methods of administering such compositions to a subject in need thereof.

### BACKGROUND OF THE INVENTION

Eyelid swelling and inflammation of the lids has both long and short-term significance in terms of histologic impact, patient quality of life, and general patient comfort. The human eyelid is made of the thinnest skin layers of the body, the most well-defined layers of tissues and muscles, and the most fragile collagen fibers. Because of these delicate skin layers, the eyelid is very susceptible to swelling, acute inflammation, and possible long-term damage.

The eyelids have several important roles that allow the eye to function as it does. They protect the eye and shield the cornea by reflexive closing. It is this mechanism that often prevents the entry of particles or foreign objects into the eye and possible damage. The lids also control the amount of light that enters the eye, just as a shutter in a camera does. They also add to the components of the tear film (via the lid margin) and maintain distribution of the smooth liquid over the eye by their spreading action during blinking. The eyelids play a very large role in maintaining not only the health of the eye, but the overall function of the ocular system. When inflammation of this crucial protection mechanism occurs, the ocular health of the individual is compromised.

Repeated stretching and damage to the lids as a result of swelling of various etiologies can cause the temporary development of sagging, drooping skin layers above and below the eye. This swelling of the lids can provide a very undesirable appearance and can even restrict the field of vision. While these signs are often only temporary, the actual damage that occurs on the physiologic and anatomic levels can eventually result in permanent changes because it accumulates with each recurrence.

This symptom of eyelid swelling is not often considered to be of primary concern when assessing ocular health, although it is a major concern for many patients, physicians and researchers. Morning eyelid swelling is very common and has both extensive social concerns in addition to concerns relating to patient health. Patients' annoyance and overall intolerance with puffy, sagging eyelids is clearly shown by the fact that eyelid surgery (229,092) and botulinum toxin injection (1,658,667) were two of the most common procedures performed by plastic surgeons in the U.S. in 2002. Despite this significant desire to reduce the presence of eyelid edema, there has been a lack of attention to the symptom. It is often classified among other signs and symptoms but is rarely a primary variable in clinical studies, as historically it has been difficult to precisely measure. Various ocular allergy medications, like olopatadine 0.1% (Patanol) begin to reduce eyelid swelling relating to allergic conjunctivitis, but there is no medication available to specifically combat this symptom directly and effectively. With such a powerful presence of so many forms in society, a treatment that directly impacts the condition of lid swelling is necessary.

### SUMMARY OF THE INVENTION

Provided are novel compositions and methods for treating and preventing eyelid swelling, particularly non-allergic eyelid swelling. In certain embodiments, novel topical ophthalmic formulations comprising an osmotically active agent and/or a vasoconstrictor and/or an astringent agent is provided. In particular the invention provides acceptable topical ophthalmic formulations comprising a combination of an osmotically active agent and/or a vasoconstrictor and/or astringent agent, which act synergistically to treat and prevent eyelid swelling. The extraordinary efficacy of these formulations is attributed to, among other things, the synergistic effect of the combination of ingredients in them. The combination of an osmotically active agent and/or a vasoconstrictor and/or an astringent agent act synergistically to treat signs and symptoms of eyelid swelling, which have never been previously contemplated to be accomplished in one product containing each of these separate ingredients.

In one embodiment, the present invention provides a target osmolarity and/or osmolality range for the formulation of an effective ophthalmic composition having an acceptable (i.e., tolerable) comfort profile, for treating and preventing eyelid swelling. To be osmotically active, the osmolarity and/or osmolality of a solution must be greater than the osmolarity and/or osmolality of its surrounding environment. Osmolarity is a measure of the osmoles of solute per liter of solution, while the osmolality is a measure of the osmoles of solute per kilogram of solvent. Molarity and osmolarity are not commonly used in osmometry because they are temperature dependent; that is, water changes its volume with temperature. One skilled in the art would readily recognize that if the concentration is very low (such as the concentrations of the composition of the invention), then the terms osmolarity and osmolality are considered equivalent and have been used interchangeably herein, as applied to the compositions of the invention.

The osmolality of the human tear film ranges from approximately 250-350 mOsm/Kg in the average human eye up to average of approximately 450 mOsm/Kg in individual suffering from ocular conditions, including without limitation, dry eye disease (with a maximum of over 700 mOsm/Kg). Therefore, in order to exert a therapeutic effect and reduce edema, the osmolality of an ophthalmic solution must be constrained by a minimum to the osmolality of the human eye environment (i.e., approximately 250 to 450 mOsm/Kg). However, with increasing osmolality comes increased discomfort upon instillation. High levels of ions activate nerve endings which can cause ocular stinging. Through comfort testing, it was herein discovered that ophthalmic solutions should have an osmolality ranging from less than 2000 mOsm/Kg, and more preferably less than 1050 mOsm/Kg to have acceptable, i.e., tolerable comfort profiles. Therefore, the target osmolality range for a drop formulated for the treatment of eyelid swelling is preferably within 200 and 2000 mOsm/Kg, preferably 250 mOsm/Kg-1500 mOsm/Kg, more preferably 260 mOsm/Kg-1250 mOsm/Kg, more preferably 265mOsm/Kg to 1200 mOsm/Kg and more preferably 400 mOsm/Kg to 1150 mOsm/Kg and more preferably 500 mOsm/Kg to 1100 mOsm/Kg.

In some embodiments, the compositions of the invention comprise an osmotically active agent including but not limited to a colloidal osmotic agent and a crystalloid osmotic agent. Crystalloid osmotic agents suitable for use in the compositions of the invention include but are not limited to sodium chloride (NaCl), dextrose, sucrose, glycerol, mannitol, sorbitol, polyethylene glycol 3350 NF, magnesium citrate and lactulose. In certain embodiments, the effective amount of the crystalloid osmotic is selected from the group consisting of: about 1% to about 10% w/v sodium chloride, about 1% to about 10% w/v dextrose, about 1% to about 20% w/v glycerol, about 1% to about 20% w/v mannitol, about 1% to about 95% w/v sucrose, and about 1% to about 95% w/v sorbitol. Preferably, the crystalloid osmotic is sodium chloride, and the effective amount is about 1% to about 10% w/v, more preferably about 2% to about 5% w/v.

Colloidal osmotic agents suitable for use in the compositions of the invention include but are not limited to: hetastarch, pentastarch, gelatin polypeptides cross-linked with urea, dextran 70, dextran 40, albumin, icodextrin, bentonite USP, MgAl silicate NF type 2A, alginic acid/sodium alginate NF, microcrystalline cellulose and CMC NF, carbomer and gellan gum.

In certain embodiments, the effective amount of the colloidal osmotic is selected from the group consisting of: about 1% to about 10% w/v hetastarch, about 1% to about 20% w/v pentastarch, about 1% to about 10% w/v dextran 70, about 1% to about 10% w/v dextran 40, about 1% to about 50% w/v albumin, and about 1% to about 50% w/v microcrystalline cellulose.

Other osmotic agents suitable for use in the methods of the invention include but are not limited to: magnesium sulfate, magnesium chloride, lithium chloride, potassium sulfate, sodium carbonate, sodium sulfite, lithium sulfate, calcium bicarbonate, sodium sulfate, calcium sulfate, potassium acid phosphate, calcium lactate, magnesium succinate, tartaric acid- and soluble carbohydrates such as raffinose, glucose, caffeine, carbomer 934P, tannic acid, ascorbic acid, dextran-40,000, inulin, menthol, polysorbate 80, and mixtures thereof. In certain embodiments, the effective amount of the osmotic is about 0.001% to about 10% w/v caffeine, about 0.001% to about 10% w/v carbomer 934P, about 0.001% to about 10% w/v tannic acid, about 0.001% to about 10% w/v ascorbic acid, about 0.001% to about 10% w/v dextran-40,000, about 0.001% to about 10% w/v inulin, about 0.001% to about 10% w/v menthol, about 0.001% to about 10% w/v polysorbate-80, or mixtures thereof.

In some embodiments, the compositions of the invention comprise a vasoconstrictor. Vasoconstrictors suitable for use in the compositions of the invention include but are not limited to naphazoline, oxymetazoline, phenylephrine, tetrahydrozoline, and other agents that are alpha receptor agonists that are vasoactive. In a preferred embodiment, the vasoconstrictor is naphazoline and the effective amount is in the range of about 0.01% to about 10% w/v, preferably about 0.01% to about 1% w/v, more preferably about 0.01% to about 0.5% w/v, even more preferably about 0.01% to about 0.2% w/v, even more preferably about 0.09% to about 0.1% w/v. In another preferred embodiment, the vasoconstrictor suitable for use in the invention is oxymetazoline, and the effective amount is in the range of about 0.01% to about 0.2% w/v, more preferably 0.01% to about 0.1% w/v, even more preferably about 0.03% to about 0.05% w/v. In yet another preferred embodiment, the vasoconstrictor suitable for use in the invention is phenylephrine and the effective amount is in the range of about 0.01% to about 10% w/v, preferably about 0.01% to about 1% w/v, more preferably about 0.01% to about 0.5% w/v, even more preferably about 0.05% to about 0.2% w/v.

In still other embodiments, the compositions of the invention comprise an astringent agent. Astringents suitable for use in the compositions of the invention include but are not limited to witch hazel, zinc sulfate, silver sulfate, plant tannins, oak bark extract, pentagalloyl glucose, alum, burow's solution, black thorn extract, bird cherry extract and natural flavanoids. Preferably, the astringent agent is witch hazel and/or zinc sulfate and the effective amount is in the range of about 0.001% to about 10% w/v, preferably about 0.01% to about 5% w/v, more preferably about 0.1% to about 1% w/v, even more preferably about 0.2% to about 0.75% w/v.

In a certain embodiment, the compositions of the invention comprise a combination of an osmotically active agent and a vasoconstrictor. In one embodiment, the osmotically active agent is NaCl or glycerol and the vasoconstrictor is naphazoline or oxymetazoline. Preferably, the sodium chloride is present in the range of about 1% to about 10% w/v, more preferably about 2% to about 5% w/v; the glycerol is present in the range of about 1% to about 30% w/v, preferably 1% to about 20% w/v, more preferably about 1% to about 10% w/v, even more preferably about 5% to about 8% w/v; the naphazoline is present in the range of about 0.01% to about 0.5% w/v, more preferably about 0.01% to about 0.2% w/v; and the oxymetazoline is present in the range of about 0.01% to about 0.2% w/v, more preferably 0.01% to about 0.1% w/v, even more preferably about 0.03% to about 0.05% w/v.

For example, the osmotically active agent is NaCl 3% w/v or glycerol 7.5% w/v, and vasoconstrictor is naphazoline 0.09% w/v or oxymetazoline 0.05% w/v. In one embodiment, the osmotic agent is glycerol 7.5% w/v and the vasoconstrictor is naphazoline 0.09% w/v. In another embodiment, the osmotic agent is glycerol 7.5% w/v and the vasoconstrictor is oxymetazoline 0.05% w/v. In still another embodiment, the osmotic agent is NaCl 3% w/v and the vasoconstrictor is naphazoline 0.09% w/v. In yet another embodiment, the osmotic agent is NaCl 3% w/v and the vasoconstrictor is oxymetazoline 0.05% w/v.

In a particular embodiment, the compositions of the invention comprise a pharmaceutically acceptable carrier and 0.9 mg/mL naphazoline hydrochloride, 30 mg/mL sodium chloride, 1 mg/mL edetate disodium, 5 mg/mL boric acid, and 0.1 mg/mL benzalkonium chloride, wherein the pH of the composition is 6.0.

In another particular embodiment, the compositions of the invention comprise a pharmaceutically acceptable carrier and 0.9 mg/mL naphazoline hydrochloride, 75 mg/mL glycerol, 1 mg/mL edetate disodium, 5 mg/mL boric acid, and 0.1 mg/mL benzalkonium chloride, wherein the pH of the composition is 6.0.

In still another particular embodiment, the compositions of the invention comprise a pharmaceutically acceptable carrier and 0.5 mg/mL oxymetzoline hydrochloride, 30 mg/mL sodium chloride, 1 mg/mL edetate disodium, 5 mg/mL boric acid, and 0.1 mg/mL benzalkonium chloride, wherein the pH of the composition is 6.0.

In yet another particular embodiment, the compositions of the invention comprise a pharmaceutically acceptable carrier and 0.5 mg/mL oxymetazoline hydrochloride, 75 mg/mL glycerol, 1 mg/mL edetate disodium, 5 mg/mL boric acid and 0.1 mg/mL benzalkonium chloride, wherein the pH of the composition is 6.0.

In some embodiments, the compositions of the invention comprise a combination of an osmotically active agent and a vasoconstrictor, wherein the osmotically active agent is selected from the group consisting of caffeine, carbomer 934P, tannic acid, ascorbic acid, dextran 40,000, inulin, mannitol, menthol, and polysorbate 80, and wherein the vasoconstrictor is selected from the group consisting of naphazoline, oxymetazoline, phenylephrine, and tetrahydrozoline.

Optionally, the osmotically active agent, and/or vasoconstrictor, and/or astringent agent is combined with various other agents, for use in treating and preventing eyelid swelling, including but not limited to additional vasoconstrictors, tear substitutes, antiallergenic agents, antihistamines, mast cell stabilizers, NSAIDs, steroids, anti-inflammatory agents, anti-oxidant agents, anti-infective agents, cholinergic agents, and combinations thereof.

The compositions of the invention may be formulated for topical administration as solutions, suspensions, oils, viscous or semi-viscous gels, emulsions, liposomes, lotions, ointments, creams, gels, salves, powders, sustained or slow release formulations or implants, eyelid lotions, or other types of solid or semi-solid compositions, and in sprayable or nebulizer form. The compositions of the invention may be formulated for acute or chronic dosing for the treatment and/or prevention of eyelid swelling.

The invention also features novel methods of treating and preventing eyelid swelling with these formulations. In some embodiments the method of treating and preventing eyelid swelling in a subject comprises topically administering a composition of the invention to the eye surface of a subject to treat and prevent eyelid swelling. In other embodiments, the method of the invention comprises topically administering a composition of the invention to the inner and/or outer eyelid of a subject to treat and prevent eyelid swelling.

In some embodiments, the method of treating and preventing eyelid swelling in a subject comprises: administering to the inner or outer eye/eyelid surface of the subject an effective amount of at least one active agent selected from the group consisting of: an osmotically active agent, a vasoconstrictor, and an astringent agent.

In another embodiment, the method of treating and preventing eyelid swelling in a subject comprises administering to the inner or outer eye/eyelid surface of the subject an effective amount of a combination of at least two agents selected from an osmotically active agent, a vasoconstrictor, and an astringent agent. In a particular embodiment, a combination of an effective amount of an osmotic agent and a vasoconstrictor is administered to the inner or outer eye/eyelid surface of the subject. For example, the osmotically active agent is NaCl or glycerol and the vasoconstrictor is naphazoline or oxymetazoline.

In one embodiment, the methods of the invention comprise administering a combination of glycerol 7.5% w/v and naphazoline 0.09% w/v to the inner or outer eye/eyelid surface of the subject for treating and preventing eyelid swelling. In another embodiment, the methods of the invention comprise administering a combination glycerol 7.5% w/v and oxymetazoline 0.05% w/v to the inner or outer eye/eyelid surface of the subject for treating and preventing eyelid swelling. In still another embodiment, the methods of the invention comprise administering a combination of NaCl 3% w/v and naphazoline 0.09% w/v to the inner or outer eye/eyelid surface of the subject for treating and preventing eyelid swelling. In yet another embodiment, the methods of the invention comprise administering a combination of NaCl 3% w/v and oxymetazoline 0.05% w/v to the inner or outer eye/eyelid surface of the subject for treating and preventing eyelid swelling.

In a particular embodiment, the methods of the invention comprise administering a combination of 0.9 mg/mL naphazoline hydrochloride, 30 mg/mL sodium chloride, 1 mg/mL edetate disodium, 5 mg/mL boric acid, and 0.1 mg/mL benzalkonium chloride, and a pharmaceutically acceptable carrier, pH 6.0, to the inner or outer eye/eyelid surface of the subject for treating and preventing eyelid swelling.

In another particular embodiment, the methods of the invention comprise administering a combination of 0.9 mg/mL naphazoline hydrochloride, 75 mg/mL glycerol, 1 mg/mL edetate disodium, 5 mg/mL boric acid, and 0.1 mg/mL benzalkonium chloride, and a pharmaceutically acceptable carrier, (overall pH=6.0), to the inner or outer eye/eyelid surface of the subject for treating and preventing eyelid swelling.

In still another particular embodiment, the methods of the invention comprise administering a combination of 0.5 mg/mL oxymetzoline hydrochloride, 30 mg/mL sodium chloride, 1 mg/mL edetate disodium, 5 mg/mL boric acid, and 0.1 mg/mL benzalkonium chloride, and a pharmaceutically acceptable carrier (overall pH=6.0), to the inner or outer eye/eyelid surface of the subject for treating and preventing eyelid swelling.

In yet another particular embodiment, the methods of the invention comprise administering a combination of 0.5 mg/mL oxymetazoline hydrochloride, 75 mg/mL glycerol, 1 mg/mL edetate disodium, 5 mg/mL boric acid and 0.1 mg/mL benzalkonium chloride, and a pharmaceutically acceptable carrier (overall pH=6.0), to the inner or outer eye/eyelid surface of the subject for treating and preventing eyelid swelling.

In some embodiments, the methods of the invention comprise administering a combination of an osmotically active agent and a vasoconstrictor, wherein the osmotically active agent is selected from the group consisting of caffeine, carbomer 934P, tannic acid, ascorbic acid, dextran 40,000, inulin, mannitol, menthol, and polysorbate 80, and wherein the vasoconstrictor is selected from the group consisting of naphazoline, oxymetazoline, phenylephrine, and tetrahydrozoline, to the inner or outer eye/eyelid surface of the subject for treating and preventing eyelid swelling.

Such formulations may be administered at an appropriate dosage depending on absorption, inactivation, and excretion rates of the drug and the delivery rate of the compound during the daytime, night-time, immediately before bedtime, and/or immediately upon awakening, to treat and prevent eyelid swelling. Such formulations may also be administered for acute or chronic use to treat and prevent eyelid swelling.

Further, the invention features a method for measuring changes in eyelid swelling using a controlled objective technique that utilizes scanning imaging technology (e.g., 3D scanning technology). Such methods enable an objective and precise quantification of daily fluctuation in lid swelling.

Even further, the invention features kits for the shipping, storage or use of the formulations, as well the practice of the methods. Other features and advantages of the invention will become apparent from the following detailed description and claims.

### BRIEF DESCRIPTION OF THE DRAWINGS

FIG. 1 contains a partial table of medical conditions that present eyelid swelling, details of such presentation for each condition and other symptoms of such conditions.

FIG. 2 depicts the effect of an osmotic agent on eyelid swelling.

FIGS. 3A-3L are line graphs depicting the results of a study using naphazoline 0.1% for treatment of morning lid swelling in 11 subjects. In each of FIGS. 3A-3L, values are represented with respect to baseline, timepoints represents time after instillation of the study drug. For each subject, the right eye (circles, also denoted as “OD”) was treated with naphazoline hydrochloride (0.1%) while the left eye (squares, also denoted as “OS”) received no treatment.

FIGS. 4A-4G are line graphs depicting the results of a study evaluating NaCl 5% ophthalmic solution for treatment of morning eyelid edema in 6 subjects. In each of FIGS. 4A-4G, values are represented with respect to baseline, error bar represents one standard error, and timepoints represents time after instillation of the study drug. For each subject, no treatment was administered in either eye at baseline, the right eye (circles, also denoted as “OD”) was treated with NaCl 5% ophthalmic solution while the left eye (squares, also denoted as “OS”) received no treatment.

FIG. 5 is a line graph depicting the results of a study comparing the efficacy of a combination of naphazoline 0.1% and NaCl 5% solution with naphazoline 0.1% or NaCl 5% individually, (and no treatment control) for treatment of morning lid swelling.

FIG. 6 is a bar graph depicting the combination of naphazoline 0.1% and NaCl 5% results of the study shown in FIG. 5.

FIG. 7A is a table summarizing the combined formulation of NaCl 5% and naphazoline 0.1% as compared to each individual component alone (column 1), tested for efficacy in reducing morning eyelid swelling, the osmolality of each test article (column 2), the percent reduction in morning eyelid swelling by the corresponding test article (column 3), the percent reduction in eyelid swelling in the control eye (no test article, column 4), the normalized percent reduction in eyelid swelling (column 5), and the standard error of deviation (column 6); FIG. 7B is a bar graph depicting the percent reduction in eyelid swelling by each test article.

FIG. 8 is a bar graph depicting the results of a study evaluating the efficacy of a combination of naphazoline hydrochloride (0.05%) dissolved in NaCl 5% ophthalmic ointment for treatment of morning eyelid swelling in 4 subjects.

FIG. 9 is a bar graph depicting the results of a study evaluating the efficacy of a combination of naphazoline hydrochloride (0.1%) dissolved in NaCl 2.5% ophthalmic solution for treatment of morning eyelid swelling in 6 subjects. Error bars represent one standard error.

FIG. 10 is a bar graph depicting the results of a study evaluating the efficacy of a combination of naphazoline hydrochloride (0.1%) in sucrose 50% solution for treatment of morning eyelid swelling in 6 subjects. Error bars represent one standard error.

FIG. 11A is a table summarizing the combined formulation of sucrose 50% and naphazoline 0.1% as compared to each individual component alone (column 1), tested for efficacy in reducing morning eyelid swelling, the osmolality of each test article (column 2), the percent reduction in morning eyelid swelling by the corresponding test article (column 3), the percent reduction in eyelid swelling in the control eye (no test article, column 4), the normalized percent reduction in eyelid swelling (column 5), and the standard error of deviation (column 6); FIG. 11B is a bar graph depicting the percent reduction in eyelid swelling by each test article.

FIG. 12 is a line graph depicting the natural progression of morning eyelid swelling in the right eye (OD), left eye (OS) and both eyes (OU) of study participants. No treatment was administered in this experiment.

FIG. 13 is a bar graph depicting the results of a study evaluating the efficacy of a topical phenylephrine 0.1% ointment for treatment of morning eyelid swelling in 6 subjects. Error bars represent one standard error.

FIG. 14A is a bar graph depicting the results of a study evaluating the efficacy of a combination of naphazoline hydrochloride (0.1%) dissolved in NaCl 5% and mannitol 12.5% ophthalmic solution for treatment of morning eyelid swelling in 6 subjects. Error bars represent one standard error; FIG. 14B is a table summarizing the combined formulation of naphazoline hydrochloride (0.1%) dissolved in NaCl 5% and mannitol 12.5% ophthalmic solution for treatment of morning eyelid swelling as compared to each individual component alone (column 1), the osmolality of each test article (column 2), the percent reduction in morning eyelid swelling by the corresponding test article (column 3), the percent reduction in eyelid swelling in the control eye (no test article, column 4), the normalized percent reduction in eyelid swelling (column 5), and the standard error of deviation (column 6); FIG. 14C is a bar graph depicting the percent reduction in eyelid swelling by each test article.

FIG. 15 is a line graph depicting the results of a study evaluating the efficacy of mannitol 12.5% ophthalmic solution for treatment of morning eyelid swelling in 6 subjects. Error bars represent one standard error.

FIG. 16A is a table summarizing the combined formulations of NaCl 5% and naphazoline 0.1%, of sucrose 50% and naphazoline 0.1%, and of NaCl 5%, mannitol 12.5% and naphazoline 0.1%, as compared to each individual component alone (column 1), tested for efficacy in reducing morning eyelid swelling, the osmolality of each test article (column 2), the percent reduction in morning eyelid swelling by the corresponding test article (column 3), the percent reduction in eyelid swelling in the control eye (no test article, column 4), the normalized percent reduction in eyelid swelling (column 5), and the standard error of deviation (column 6); FIG. 16B is a bar graph depicting the percent reduction in eyelid swelling by each test article.

FIG. 17 is a bar graph depicting the results of a study evaluating the efficacy of sucrose 50% ophthalmic solution for treatment of morning eyelid swelling in six subjects. Error bars represent one standard of error.

FIG. 18A is a table indicating the osmolality and mean comfort levels of various ophthalmic solutions. FIG. 18B is a line graph depicting the correlation between osmolality and comfort (on a scale of 0-10, (0 indicating most comfort, 10 indicating most discomfort) for six different ophthalmic formulations ranging in osmolality from approximately 800 mOsm/Kg to 2400 mOsm/Kg.

FIG. 19 is a bar graph indicating the mean comfort levels of various ophthalmic formulations (“Oxy” denotes oxymetazoline; “Naph” denotes naphazoline).

FIG. 20 is a line graph depicting mean baseline lid swelling scores for twenty subjects, based on a subjective regional/global lid swelling scale. Lid swelling was assessed in the evening and in the following morning upon awakening (baseline), followed by ten minute intervals for up to one hour.

FIG. 21 is a line graph depicting mean global scores of evening and morning lid swelling over a 6 day period for nineteen subjects. Global lid swelling was subjectively assessed on a scale of 0-3 (0=none, 3=definite swelling)

FIG. 22 is a line depicting mean lid swelling in region 1 of the human eyelid before (baseline) and immediately after (time=0) instillation of one drop of naphazoline 0.09%/NaCl 3% in one eye, and placebo in the fellow eye (N=12). Lid swelling was assessed over a 60 minute period.

FIG. 23 is a line graph depicting mean lid swelling in region 2 of the human eyelid before (baseline) and immediately after (time=0) instillation of one drop of naphazoline 0.09%/NaCl 3% in one eye, and placebo in the fellow eye (N=10). Lid swelling was assessed over a 60 minute period.

FIG. 24 is a line graph depicting mean lid swelling in region 3 of the human eyelid before (baseline) and immediately after (time=0) instillation of one drop of naphazoline 0.09%/NaCl 3% in one eye, and placebo in the fellow eye (N=15). Lid swelling was assessed over a 60 minute period.

FIG. 25 is a line graph depicting mean lid swelling in region 4 of the human eyelid before (baseline) and immediately after (time=0) instillation of one drop of naphazoline 0.09%/NaCl 3% in one eye, and placebo in the fellow eye (N=16). Lid swelling was assessed over a 60 minute period.

FIG. 26 is a line graph depicting mean global lid swelling before (baseline) and after instillation of one drop of naphazoline 0.09%/NaCl 3% in one eye, and placebo in the fellow eye (N=10).

FIG. 27 is a bar graph depicting mean comfort scores for naphazoline 0.09%/NaCl 3% ophthalmic formulation and placebo.

FIG. 28 is a line graph depicting mean global lid swelling scores before (baseline) and immediately after (time=0) instillation of one drop of naphazoline 0.09%/NaCl 3% in one eye, and oxymetazoline 0.05%/NaCl 3% in the follow eye. Lid swelling was assessed over a 6 hour period (360 minutes).

FIG. 29 is a line graph depicting mean global lid swelling scores before (baseline) and immediately after (time=0) instillation of one drop of naphazoline 0.09%/glycerol 7.5% in one eye, and oxymetazoline 0.05%/glycerol 7.5% in the fellow eye. Lid swelling was assessed over a 6 hour period (360 minutes).

FIG. 30 is a line graph comparing mean global lid swelling scores before (baseline) and immediately after (time=0) instillation of naphazoline 0.09%/NaCl 3%, oxymetazoline 0.05%/NaCl 3%, naphazoline 0.09%/glycerol 7.5% and oxymetazoline 0.05%/glycerol 7.5%. Lid swelling was assessed over a 6 hour period (360 minutes).

FIG. 31 is a bar graph comparing mean comfort scores for naphazoline 0.09%/NaCl 3%, oxymetazoline 0.05%/NaCl 3%, naphazoline 0.09%/glycerol 7.5% and oxymetazoline 0.05%/glycerol 7.5%.

### DETAILED DESCRIPTION OF THE INVENTION

### 1. Definitions

For convenience, before further description of the present invention, certain terms employed in the specification, examples, and appended claims are collected here. These definitions should be read in light of the remainder of the disclosure and understood as by a person of skill in the art.

As used herein, the term “acceptable comfort profile” refers to the tolerability of an ophthalmic formulation when administered to the eye, wherein the benefit of administering such ophthalmic formulation to the eye to alleviate, soothe, treat, and/or prevent an ocular condition outweighs the risk of any discomfort associated with administration of said formulation to the eye, such as to increase patient compliance in administering said ophthalmic formulation to the eye.

The term “antiallergenic agent” refers to a molecule or composition that treats ocular allergy or reduces a symptom of ocular allergy. Examples of antiallergenic agents include, but are not limited to, “antihistamines” or drugs which block histamine from binding to the histamine receptors, “mast cell stabilizers” or drugs that block the release of histamine and other substances from the mast cell, “drugs with multiple modes of action” or drugs that are antiallergenic agents having multiple modes of action (e.g. drugs that are antihistamines and mast cell stabilizers, drugs with antihistamine, mast cell stabilizing and anti-inflammatory activity, etc.), and nonsteroidal anti-inflammatory drugs or “NSAIDs” and steroids.

The term “aqueous” typically denotes an aqueous composition wherein the carrier is to an extent of >50%, more preferably >75% and in particular >90% by weight water.

The phrase “effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to eliminate, reduce or maintain (e.g., prevent the spread of) eyelid swelling, or prevent or treat eyelid swelling. The effective amount may vary depending on such factors as the disease or condition being treated, the particular composition being administered, or the severity of the disease or condition. One of skill in the art may empirically determine the effective amount of a particular agent without necessitating undue experimentation. For the treatment of eyelid swelling, an effective amount preferably refers to the amount of a therapeutic agent that reduces eyelid swelling by at least 2%, at least 5%, at least 10%, at least 15%, at least 20%, at least 30%, at least 35%, at least 40%, at least 45%, at least 50%, at least 60%, at least 65%, at least 70%, at least 75%, at least 80%, at least 85% at least 90%, at least 95%, or at least 100%, as determined by a ruler, subjective scales assessing eyelid swelling (for example, but not limited to, subjective clinical scales that determine swelling as mild, moderate, severe, or 0, 1, 2, or 3, or other appropriate scale), and/or 3D scanning technology.

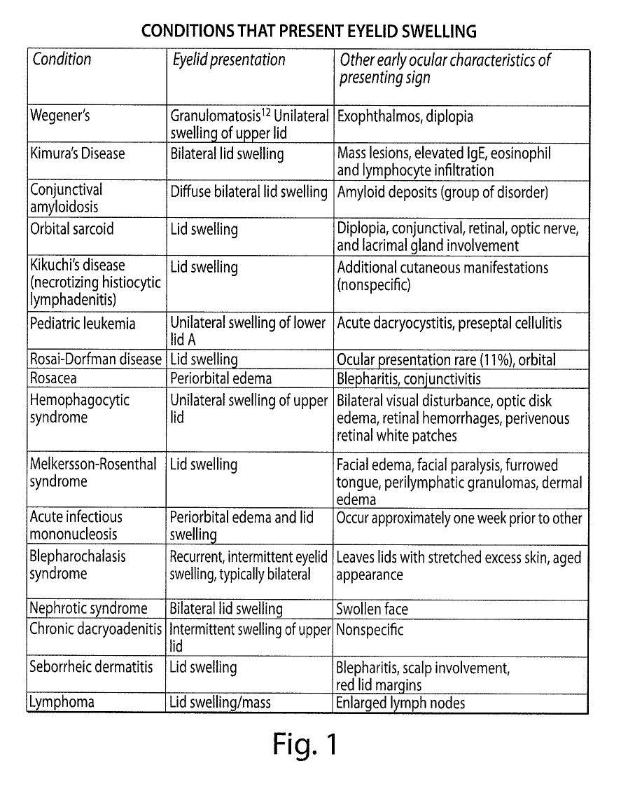

The term “eyelid swelling” refers to any non-allergic or allergic condition comprising the swelling or inflammation of the eyelids, including periorbital edema. For example without limitation, all of the conditions listed in FIG. 1 are encompassed within the term non-allergic “eyelid swelling.” Thus, “eyelid swelling” as defined herein encompasses any cause of eyelid swelling ranging from uncommon disorders like blepharochalasis, to the more common dermatochalasis, characterized by “bags under the eyes.” In addition to these swelling infections, there are many other non-allergic conditions that can result in swelling of the eyelids, including, but not limited to, rosacea, dermatitis caused by cosmetics or topical pharmaceuticals, lymphoma, renal and endocrine dyfunctions (thyroid), and even trichinosis, an infectious disease for which the chronic periocular edema can be a very useful diagnostic sign. More common causes of eyelid swelling include age, alcohol use, computer use, reading, fatigue and diurnal variations (morning eyelid swelling.) Morning eyelid swelling occurs overnight and results in eyelid swelling in the morning upon awakening. Further, ocular allergies are one of the most common causes of eyelid inflammation, with almost 20% of the general population being affected. In this case, the array of pre-formed mediators released as a result of IgE-stimulated mast cell degranulation are responsible for the clinical signs and symptoms of an allergic reaction causing vasodilation of the vasculature and leakage of fluid from the blood stream to the tissue.

The term “hyperosmotic solution” as used herein refers to any solution having an osmolality greater than another fluid, e.g., that comprises a higher concentration of osmotically active components than the other fluid.

The term “ocular allergy” as used herein refers to any allergic disease of the eye. Examples of such ocular allergies include but are not limited to seasonal/perennial allergic conjunctivitis, vernal keratoconjunctivitis, giant papillary conjunctivitis, perennial allergic conjunctivitis and atopic keratoconjunctivitis. The signs and symptoms of ocular allergies include chemosis, eye itching, redness, tearing, and eyelid swelling.

The term “osmotically active agent” refers to a water-attracting agent, e.g., a hygroscopic, hydroscopic or other agent, which drives the osmotic flow in a hyperosmotic solution. To be osmotically active, the osmolality of a solution must be greater than the osmolality of its surrounding environment.

A “patient,” “subject,” or “host” to be treated by the subject method refers to either a human or non-human animal, such as primates, mammals, and vertebrates.

The phrase “pharmaceutically acceptable” is art-recognized and refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerol, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) aqueous solutions, suspensions, ointments, and (22) other non-toxic compatible substances employed in pharmaceutical formulations.

The term “pharmaceutically acceptable salts” is art-recognized, and refers to relatively non-toxic, inorganic and organic acid addition salts of compositions of the present invention or any components thereof, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include but are not limited too the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

The term “preventing,” when used in relation to a condition, is art-recognized, and refers to administration of a composition which reduces the frequency of, or delays the onset of, symptoms of a medical condition in a subject relative to a subject which does not receive the composition.

The term “treating” is an art-recognized term which refers to curing and/or ameliorating at least one symptom of any condition or disease by administering one or more diagnostic, therapeutic, or prophylactic agents, including but not limited to ocular agents such as osmotically active agents, vasoconstrictors, astringent agents, and a combination thereof.

The term “vasoconstrictors” refers to any drug or agent that constricts blood vessels, including but not limited to agents that act on alpha-1 receptors in smooth muscle tissues.

### 2. Eyelid Swelling

Eyelid swelling can occur as a result of a number of different pathological conditions including allergy, infection, mild irritation/inflammation, trauma, and morning eyelid swelling. Morning eyelid swelling occurs as a result of lost tissue turgor and inflammation. As the individual ages, the skin surrounding the eyelids loses its elasticity. The collagen fibers that provide the dermis with rigidity and elasticity begin to break down, a natural process that can be exacerbated by excessive exposure to sunlight or other destructive environmental stimuli such as smoke. In addition, underlying orbital fat is broken down, leading again to the development of flaccid, empty appearing tissue, or lost tissue turgor.

When an individual sleeps in a horizontal position, fluid leaks out of the underlying vasculature into the empty, structureless tissue surrounding the eyes, in particular the lower eyelid. This may be caused by accumulation of inflammatory mediators in the tear film and conjunctiva. The lost elasticity of the dermis allows the superficial eyelid tissue to expand with the increase in fluid. When the individual awakens, the eyelids appear puffy and swollen as a result of the excess fluid that has drained into the broken down eyelid tissue. Variable fluid accumulation may occur in the tissues overlying the orbital bone at the outer corner of the lower eyelid. This fluid may appear dark blue, or purple in color, contributing the appearance or tired, baggy eyes. After an individual awakens and assumes an upright position, eyelid swelling gradually decreases as fluid drains out of the eyelid tissue. However, this process can take a considerable amount of time.

Eyelid swelling and periorbital edema is distinguishable from other types of ocular edema, such as corneal edema. As described, eyelid swelling develops as a result of fluid leaking from the underlying vasculature within the orbital and periorbital region. In contrast, the cornea does not contain blood vessels. Corneal edema typically results from abnormal intraocular pressure, electrolyte imbalance within the corneal stroma, and/or the presence of an active metabolic pump in the endothelium, each of which drives fluid into the cornea.

As such, a pharmaceutical composition formulated for ophthalmic use comprising an effective amount of an active agent selected from an osmotically active agent, a vasoconstrictor, an astringent agent, or combinations thereof, which is instilled directly into the eye is effective to treat eyelid swelling by “drying out” the underlying vasculature to treat and prevent leakage into the eyelid tissue and periorbital region. A pharmaceutical composition formulated for ophthalmic use comprising an effective amount of an active agent selected from an osmotically active agent, a vasoconstrictor, an astringent agent, or combinations thereof, which is applied to the inner and/or outer surface of the ocular surface/eyelid is also effective to treat and prevent eyelid swelling.

### 3. Pharmaceutical Compositions

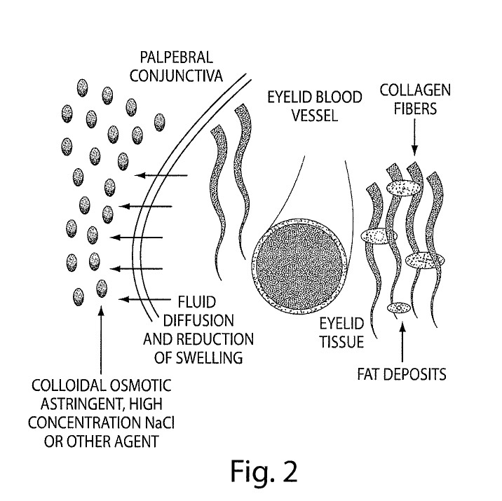

Featured are novel topical pharmaceutical compositions comprising an effective amount of one or more active agents in a pharmaceutically acceptable carrier for the treatment and prevention of eyelid swelling and periorbital edema. The one or more active agents may include, but are not limited to, osmotically active agents, vasoconstrictors, astringent agents, or combinations thereof. The astringent or osmotically active agent serves to pull fluid out of swollen or inflamed tissue (FIG. 2), while a vasoconstrictor serves to prevent additional leakage from the underlying vasculature into the eyelid tissue. In a particular embodiment, the pharmaceutical compositions of the invention comprise at least two active agents, including but not limited to osmotically active agents, vasoconstrictors, astringent agents, or combinations thereof.

In a preferred embodiment, the pharmaceutical compositions of the invention are formulated to an osmolality of 200 and 2000 mOsm/Kg, preferably 250 mOsm/Kg-1500 mOsm/Kg, more preferably 260 mOsm/Kg-1250 mOsm/Kg, more preferably 265 mOsm/Kg to 1200 mOsm/Kg, and more preferably 400 mOsm/Kg to 1150 mOsm/Kg and more preferably 500 mOsm/Kg to 1100 mOsm/Kg. Such formulations provide a drop with an acceptable comfort profile when instilled in the eye.

In one embodiment, the active agent is an osmotically active agent. In certain embodiments, the pharmaceutical composition comprises a hyperosmotic solution containing an osmotically active agent. Hyperosmotic solutions contain a higher concentration of electrolytes than that found in surrounding environments.

In certain embodiments, the osmotically active agent is a crystalloid osmotic agent. Examples of crystalloid osmotics include, but are not limited to, sodium chloride (NaCl), dextrose, glycerol, mannitol, sorbitol, sucrose, polyethylene glycol 3350 NF, magnesium citrate and lactulose.

In certain embodiments, the crystalloid osmotic agent is mannitol. Mannitol is a sugar alcohol form of mannose that occurs naturally in many fruits and vegetables.

In other embodiments, the crystalloid osmotic agent is glycerol. Glycerol is obtained from fats and oils as a byproduct of saponification and is frequently used as a solvent for many ophthalmic products and as a component of a variety of products including cosmetics, soaps, and lubricants.

In a particular embodiment, the crystalloid osmotic agent is sodium chloride (solution, gel, suspension, or other pharmaceutically acceptable vehicle). In another particular embodiment, the crystalloid osmotic agent is glycerol (solution, gel, suspension, or other pharmaceutically acceptable vehicle).

In still other embodiments, the crystalloid osmotic agent is dextrose. Dextrose is approved for injection in adults and pediatric patients as a source of electrolytes, calories and water for hydration.

In still other embodiments, the crystalloid osmotic agent is polyethylene glycol 3350 NF.

In still other embodiments, the crystalloid osmotic agent is magnesium citrate.

In still other embodiments, the crystalloid osmotic agent is lactulose. Lactulose is a synthetic sugar.

In certain embodiments, the osmotically active agent is a colloidal osmotic. Examples of colloidal osmotics include, but are not limited to, hetastarch, pentastarch, gelatin polypeptides cross-linked with urea, dextran 70, dextran 40, albumin, icodextrin, bentonite USP, MgAl silicate NF type 2A, alginic acid/sodium alginate NF, microcrystalline cellulose and CMC NF, carbomer and gellan gum.

In certain embodiments, the colloidal osmotic agent is hetastarch. Hetastarch is a plasma expander indicated for treatment of shock due to fluid loss.

In still other embodiments, the colloidal osmotic agent is pentastarch. Like hetastarch, pentastarch is a plasma expander indicated for treatment of shock due to fluid loss.

In still other embodiments, the colloidal osmotic agent is a combination product of gelatin polypeptides cross linked with urea.

In still other embodiments, the colloidal osmotic agent is Dextran 70.

In other embodiments, the colloidal osmotic agent is Dextran 40. Like Dextran 70, Dextran 40 is indicated for fluid replacement in shock.

In still other embodiments, the colloidal osmotic agent is albumin.

In still other embodiments, the colloidal osmotic agent is Icodextrin. Icodextran is a sucrose derivative that is frequently used for osmotic applications as a substitute for glucose.

In still other embodiments the colloidal osmotic agent is MgAl Silicate NF Type 2A.

In still other embodiments the colloidal osmotic agent is alginic acid. Alginic acid is a viscous gum that is isolated from seaweed and can be used as an osmotic agent.

In still other embodiments, the colloidal osmotic agent is carboxymethylcellulose sodium (CMC) NF.

In still other embodiments, the colloidal osmotic agent is gellan gum.

In still other embodiments, the colloidal osmotic is sodium carbomer.

In still other embodiments, the colloidal osmotic agent is microcrystalline cellulose.

There are fundamental differences between colloids and crystalloids in their formulation. Crystalloids are predominately based on a solution of sterile water with added electrolytes. Crystalloids come in a variety of formulations, from those that are hypotonic to plasma to those that are isotonic or hypertonic. Colloids are often based on crystalloid solutions, thus containing water and electrolytes, but have the added component of a colloidal substance (e.g., a suspension of particles smaller than one millimicron in diameter that does not freely diffuse across a semipermeable membrane).

Other exemplary osmotically active agents contemplated for use in the pharmaceutical compositions of the invention include compounds such as magnesium sulfate, magnesium chloride, lithium chloride, potassium sulfate, sodium carbonate, sodium sulfite, lithium sulfate, calcium bicarbonate, sodium sulfate, calcium sulfate, potassium acid phosphate, calcium lactate, magnesium succinate, tartaric acid- and soluble carbohydrates such as raffinose, glucose, caffeine, carbomer 934P, tannic acid, ascorbic acid, dextran-40,000, inulin, menthol, polysorbate 80, and mixtures thereof. In certain embodiments, the effective amount of the osmotic is selected from the group consisting of: about 0.001% to about 10% w/v caffeine, about 0.001% to about 10% w/v carbomer 934P, about 0.001% to about 10% w/v tannic acid, about 0.001% to about 10% w/v ascorbic acid, about 0.001% to about 10% w/v dextran-40,000, about 0.001% to about 10% w/v inulin, about 0.001% to about 10% w/v menthol, about 0.001% to about 10% w/v polysorbate-80, or mixtures thereof.

In another embodiment, the active agent is an astringent agent (that is, an agent that among other things, shrinks tissue). Examples of astringent agents contemplated for use in the topical pharmaceutical compositions of the invention include, but are not limited to, witch hazel, zinc sulfate, silver sulfate, plant tannins, oak bark extract, pentagalloyl glucose, alum, burow's solution, black thorn extract, bird cherry extract and natural flavanoids.

In a particular embodiment, the astringent is witch hazel. Witch hazel is an isolate from an herb found in central and southern Europe.

In another particular embodiment, the astringent agent is zinc sulfate.

In still another particular embodiment, the astringent is silver sulfate.

In yet another embodiment, the active agent is a vasoconstrictor. In certain embodiments, the vasoconstrictor is an alpha-1 adrenergic agonist. In other embodiments, the vasoconstrictor is any agent that decreases the diameter of the blood vessel and thus prevents leakage. Alpha-1 adrenergic agonists contemplated for use in the topical pharmaceutical compositions of the invention include but are not limited to naphazoline, oxymetazoline, phenylephrine, and tetrahydrozoline. In a particular embodiment, the vasoconstrictor contemplated for use in the invention is naphazoline, and the effective amount is in the range of about 0.01% to about 10% w/v, preferably about 0.01% to about 1% v, more preferably about 0.01% to about 0.5% w/v, even more preferably about 0.01% to about 0.2% w/v, even more preferably about 0.09% to about 0.1% w/v. In another particular embodiment, the vasoconstrictor contemplated for use in the invention is oxymetazoline, and the effective amount is in the range of about 0.01% to about 0.2% w/v, more preferably 0.01% to about 0.1% w/v, even more preferably about 0.03% to about 0.05% w/v. In yet another particular embodiment, the vasoconstrictor contemplated for use in the invention is phenylephrine and the effective amount is in the range of about 0.01% to about 10% w/v, preferably about 0.01% to about 1% w/v, more preferably about 0.01% to about 0.5% w/v, even more preferably about 0.05% to about 0.2% w/v.

In a certain embodiment, the pharmaceutical composition of the invention comprises both a vasoconstrictor and an osmotically active agent. In a particular embodiment, the pharmaceutical composition of the invention comprises both naphazoline and NaCl. In another particular embodiment, the pharmaceutical composition of the invention comprises both oxymetazoline and NaCl. In yet another particular embodiment, the pharmaceutical composition of the invention comprises both naphazoline and glycerol. In still another particular embodiment, the pharmaceutical composition of the invention comprises both oxymetazoline and glycerol. The extraordinary efficacy of such formulations is attributed to, among other things, the synergistic effect of the combination of ingredients in them, as described in the Examples below.

The effective amount of an active agent may be present in the composition at a dose in the range of about 0.001% to about 100.0% w/v. For example, the effective amount of each active agent may be in the range of about 0.001% to about 0.01% w/v, of about 0.01% to about 0.100% w/v, of about 0.100% to about 1.0% w/v, of about 1.00% to about 10.00% w/v, or of about 10% to about 100% w/v.

One of ordinary skill in the art will recognize that the effective amount of an active agent present in the formulations of the invention will vary depending on the nature of the active agent(s) used, depending on factors including but not limited to absorption, inactivation, and excretion rates of the drug, the delivery rate of the compound, and the one or more combinations of agents. For example, an effective amount of sodium chloride is in the range of about 1% to about 10% w/v, preferably about 1% to about 6% w/v, more preferably about 2% to about 5% w/v. An effective amount of dextrose is in the range of about 1% to about 10% w/v, preferably about 1% to about 6% w/v, more preferably about 2% to about 5% w/v. An effective amount of sucrose is about 1% to about 95% w/v, preferably about 10% to about 90% w/v, more preferably about 20% to about 80% w/v, even more preferably about 30% to about 70% w/v. An effective amount of glycerol is in the range of about 1% to about 30% w/v, preferably 1% to about 20% w/v, more preferably about 1% to about 10% w/v, even more preferably about 5% to about 8% w/v. An effective amount of mannitol is in the range of about 1% to about 30% w/v, preferably about 1% to about 20% w/v, more preferably about 10% to about 15% w/v. An effective amount of sorbitol is in the range of about 1% to about 100% w/v, preferably about 10% to about 90% w/v, more preferably about 20% to about 80% w/v, even more preferably about 30% to about 70% w/v. An effective amount of hetastarch is in the range of about 1% to about 20% w/v, preferably about 1% to about 10% w/v, more preferably about 4% to about 6% w/v. An effective amount of pentastarch is in the range of about 1% to about 20% w/v, preferably about 5% to about 15% w/v, more preferably about 5% to about 10% w/v. An effective amount of dextran 70 is in the range of about 1% to about 20% w/v, preferably about 1% to about 10% w/v, more preferably about 4% to about 6% w/v. An effective amount of dextran 40 is in the range of about 1% to about 20% w/v, preferably about 1% to about 10% w/v, more preferably about 4% to about 6% w/v. An effective amount of albumin is in the range of about 10% to about 50% w/v, preferably about 15% to about 30% w/v, more preferably about 20% to 30% w/v albumin.

Solid solutes, present initially in excess, can be in any suitable physical form such as particles, crystals, pellets, tablets, strips, film; granules and the like.

In certain embodiments, the pharmaceutical compositions of the invention comprise combinations of one or more active agents selected from an osmotic agent, a vasoconstrictor, and/or an astringent, and an effective amount of another agent(s), such as an additional vasoconstrictor, tear substitute, antiallergenic agent, antihistamine, mast cell stabilizer, NSAID, steroid, anti-inflammatory agent, anti-oxidant agent, anti-infective agent, cholinergic agent, or combinations thereof. The combinations of agents may act synergistically to decrease eyelid swelling.

Exemplary vasoconstrictors contemplated for use in the pharmaceutical compositions of the invention include, but are not limited to, naphazoline, antolazine, tetrahydrozoline, oxymetazoline and phenylephrine. Vasoconstrictors may additionally act as decongestants, in addition to reducing eyelid swelling. In certain embodiments, the effective amount of vasoconstrictor is in the range of about 0.01% to about 10% w/v, preferably about 0.01% to about 1% w/v, more preferably about 0.01% to about 0.5% w/v, even more preferably about 0.01% to about 0.2% w/v. In a particular embodiment, the vasoconstrictor contemplated for use in the invention is naphazoline, and the effective amount is in the range of about 0.01% to about 10% w/v, preferably about 0.01% to about 1% w/v, more preferably about 0.01% to about 0.5% w/v, even more preferably about 0.01% to about 0.2% w/v, even more preferably about 0.09% to about 0.1% w/v. In another particular embodiment, the vasoconstrictor contemplated for use in the invention is oxymetazoline, and the effective amount is in the range of about 0.01% to about 0.2% w/v, more preferably 0.01% to about 0.1% w/v, even more preferably about 0.03% to about 0.05% w/v. In yet another particular embodiment, the vasoconstrictor contemplated for use in the invention is phenylephrine and the effective amount is in the range of about 0.01% to about 10% w/v, preferably about 0.01% to about 1% w/v, more preferably about 0.01% to about 0.5% w/v, even more preferably about 0.05% to about 0.2% w/v.

A variety of tear substitutes are known in the art and could be used in the compositions of the invention, including but not limited to: polyols such as, glycerol, glycerol, polyethylene glycol 300, polyethylene glycol 400, polysorbate 80, propylene glycol, and ethylene glycol, polyvinyl alcohol, povidone, and polyvinylpyrrolidone; cellulose derivatives such hydroxypropyl methylcellulose (also known as hypromellose), carboxy methylcellulose sodium, hydroxypropyl cellulose, hydroxyethyl cellulose, and methylcellulose; dextrans such as dextran 70; water soluble proteins such as gelatin; carbomers such as carbomer 934P, carbomer 941, carbomer 940 and carbomer 974P; and gums such as HP-guar. Many such tear substitutes are commercially available, which include, but are not limited to cellulose esters such as Bion Tears®, Celluvisc®, Genteal®, OccuCoat®, Refresh®, Teargen Il®, Tears Naturale®, Tears Naturale 118®, Tears Naturale Free®, and TheraTears®; and polyvinyl alcohols such as Akwa Tears®, HypoTears®, Moisture Eyes®, Murine Lubricating®, and Visine Tears®. Tear substitutes may also be comprised of paraffins, such as the commercially available Lacri-Lube® ointments. Other commercially available ointments that are used as tear substitutes include Lubrifresh PM®, Moisture Eyes PM® and Refresh PM®.

In a preferred embodiment, the tear substitute, or one or more components thereof, is an aqueous solution having a viscosity in a range which optimizes efficacy of supporting the tear film while minimizing blurring, lid caking, etc. Preferably, the viscosity of the tear substitute, or one or more components thereof, ranges from 30-150 centipoise (cpi), preferably 30-130 cpi, more preferably 50-120 cpi, even more preferably 60-115 cpi (or any specific value within said ranges). In a particular embodiment, the viscosity of the tear substitute, or one or more components thereof, is about 70-90 cpi, or any specific value within said range (for example without limitation, 85 cpi).

Viscosity of the ophthalmic formulations of the invention may be measured according to standard methods known in the art, such as use of a viscometer or rheometer. One of ordinary skill in the art will recognize that factors such as temperature and shear rate may effect viscosity measurement. In a particular embodiment, viscosity of the ophthalmic formulations of the invention is measured at 20° C.+/−1° C. using a Brookfield Cone and Plate Viscometer Model VDV-III Ultra⁺ with a CP40 or equivalent Spindle with a shear rate of approximately apprx. 22.50+/−apprx 10 (1/sec), or a Brookfield Viscometer Model LVDV-E with a SC4-18 or equivalent Spindle with a shear rate of approximately 26+/−apprx 10 (1/sec)).

In some embodiments, the tear substitute, or one or more components thereof is buffered to a pH 5.0 to 9.0, preferably pH 5.5 to 8.5, more preferably pH 6 to 8 (or any specific value within said ranges), with a suitable salt (e.g., phosphate salts). In some embodiments, the tear substitute further comprises one or more ingredients, including without limitation, glycerol, propyleneglycerol, glycine, sodium borate, magnesium chloride, and zinc chloride.

In one preferred embodiment of the invention, the tear substitute comprises hydroxypropylmethyl cellulose. For example, without limitation, a tear substitute which comprises hydroxypropyl methyl cellulose is GenTeal® lubricating eye drops. GenTeal® (CibaVision-Novartis) is a sterile lubricant eye drop containing hydroxypropylmethyl cellulose 3 mg/g and preserved with sodium perborate. Other examples of an HPMC-based tear are provided.

In another preferred embodiment, the tear substitute comprises carboxymethyl cellulose sodium. For example, without limitation, the tear substitute which comprises carboxymethyl cellulose sodium is Refresh® Tears. Refresh® Tears is a lubricating formulation similar to normal tears, containing a, mild non-sensitizing preservative, stabilised oxychloro complex (Purite™), that ultimately changes into components of natural tears when used.

Exemplary NSAIDs suitable for use in the compositions of the invention include but are not limited to, amfenac, propionic acids such as naproxen, flurbiprofen, oxaprozin, ibuprofen, ketoprofen, fenoprofen; ketorolac tromethamine (Acular®) (and the other compounds described as being opthalmologically effective in U.S. Pat. No. 4,454,151 to Waterbury, issued Jun. 12, 1984, the pertinent portions of which are incorporated herein by reference); acetic acid derivatives such as sulindac, indomethacin, and etodolac; phenylacetic acids such as diclofenac (Voltaren®) (and the other compounds described as being opthalmologically effective in U.S. Pat. No. 4,960,799 to Nagy, issued Oct. 2, 1990, the pertinent portions of which are incorporated herein by reference), bromfenac, and suprofen; arylacetic prodrugs such as nepafenac; salicyclic acids, such as aspirin, salsalate, diflunisal, choline magnesium trisalicylate (CMT); para-aminophenol derivatives such as acetaminophen; naphthylalkanones such as nabumetone; enolic acid derivatives such as piroxicam and meloxicam; femanates such as mefenamic acid, meclofenamate and flufenamic acid; pyrroleacetic acids such as tolmetin; and pyrazolones such as phenylbutazone; COX-2 selective inhibitors such as celecoxib, valdecoxib, parecoxib, etoricoxib, and luaricoxib; including all esters and pharmaceutically acceptable salts thereof.

Exemplary antihistamines include, but are not limited to, pheniramine, antazoline, emedastine difumarate, ebastine, carebastine, levocabastine, cetirizine, and pharmaceutically active salts thereof.

Exemplary mast cell stabilizers include, but are not limited to, nedocromil, lodoxamide, pemirolast, cromolyn, cromolyn sodium, and pharmaceutically active salts thereof.

Exemplary drugs with multiple modes of action include, but are not limited to, azelastine, epinastine, olopatadine, ketotifen fumarate, bilastine, bepotastine, mizolastine and pharmaceutically active salts thereof.

The one or more active agents of the pharmaceutical compositions may be in the form of a pharmaceutically acceptable salt.

The pharmaceutical compositions may be formulated for topical administration as solutions, suspensions, oils, viscous or semi-viscous gels, emulsions, liposomes, lotions, ointments, creams, gels, salves, powders, and sustained or slow release, as well as eyelid lotion, or other types of solid or semi-solid compositions, including formulations described in U.S. Pat. No. 6,806,364. The composition may also be topically administered in a sprayable or nebulizer form.

Preferably, the pharmaceutical compositions are gels for controlled- or sustained-release of one or more pharmaceutically active agents (e.g., an osmotically active agent or vasoconstrictor, or a combination thereof). The formulation may be an in situ gellable aqueous formulation. Such a formulation comprises a gelling agent in a concentration effective to promote gelling upon contact with the eye or with lacrimal fluid in the exterior of the eye. Suitable gelling agents include, but are not limited to, thermosetting polymers such as tetra-substituted ethylene diamine block copolymers of ethylene oxide and propylene oxide (e.g., poloxamine); polycarbophil; and polysaccharides such as gellan, carrageenan (e.g., kappa-carrageenan and iota-carrageenan), chitosan and alginate gums.

The phrase “in situ gellable” as used herein embraces not only liquids of low viscosity that form gels upon contact with the eye or with lacrimal fluid in the exterior of the eye, but also more viscous liquids such as semi-fluid and thixotropic gels that exhibit substantially increased viscosity or gel stiffness upon administration to the eye. Although it is preferred that such a formulation exhibit further increase in viscosity or gel stiffness upon administration, this is not absolutely required if the initial gel is sufficiently resistant to dissipation by lacrimal drainage to provide the effective residence time specified herein.

Sustained release ophthalmic formulations of highly viscous gels have been described in U.S. Pat. Nos. 4,271,143 and 4,407,792. Further, U.K. Patent Application GB 2007091A describes an ophthalmic composition in the form of a gel comprising an aqueous solution of a carboxyvinyl polymer, a water-soluble basic substance and an ophthalmic drug. Alternatively, U.S. Pat. No. 4,615,697 discloses a controlled release composition and method of use based on a bioadhesive and a treating agent.

In certain embodiments, the pharmaceutical compositions according to the present invention may be formulated as hyperosmotic solutions for topical administration. Aqueous solutions are easy to formulate, and are easily administered by a patient by means of instilling one to two drops of the solutions in the affected eyes.

Any of a variety of carriers may be used in the formulations of the present invention including water, mixtures of water and water-miscible solvents, such as, but not limited to, C1- to C7-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. The concentration of the carrier is, typically, from 1 to 100,000 times the concentration of the active ingredient.

Additional ingredients that may be included in the formulation include tonicity enhancers, preservatives, solubilizers, non-toxic excipients, demulcents, sequestering agents, pH adjusting agents, co-solvents and viscosity building agents.

For the adjustment of the pH, preferably to a physiological pH, buffers may be especially useful. The pH of the present solutions should be maintained within the range of 4.0 to 8.0, more preferably about 4.0 to 6.0, more preferably about 6.5 to 7.8. Suitable buffers may be added, such as, but not limited to, boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Generally, buffers will be used in amounts ranging from about 0.05 to 2.5 percent by weight, and preferably, from 0.1 to 1.5 percent.

Tonicity is adjusted if needed typically by tonicity enhancing agents. Such agents may, for example be of ionic and/or non-ionic type. Examples of ionic tonicity enhancers are, but are not limited to, alkali metal or earth metal halides, such as, for example, CaCl₂, KBr, KCl, LiCl, NaI, NaBr or NaCl, Na₂SO4 or boric acid. Non-ionic tonicity enhancing agents are, for example, urea, glycerol, sorbitol, mannitol, propylene glycol, or dextrose. These agents may also serve as the active agents in certain embodiments. In certain embodiments, these agents may also serve to adjust osmolality.

To be osmotically active, the osmolality of a solution must be greater than the osmolality of its surrounding environment. The osmolality of the human tear film ranges from approximately 250-350 mOsm/Kg in the average human eye up to average of approximately 450 mOsm/Kg in individual suffering from ocular conditions, including without limitation, dry eye disease (with a maximum of over 700 mOsm/Kg). Therefore, in order to exert a therapeutic effect and reduce edema, the osmolality of an ophthalmic solution must be constrained by a minimum to the osmolality of the human eye environment (i.e., approximately 250 to 450 mOsm/Kg). However, with increasing osmolality comes increased discomfort upon instillation. High levels of ions activate nerve endings which can cause ocular stinging. Through comfort testing, it was discovered that ophthalmic solutions should have an osmolality ranging from less than 2000 mOsm/Kg, and more preferably less than 1050 mOsm/Kg to have acceptable, i.e., tolerable comfort profiles. Therefore, the target osmolality range for a drop formulated for the treatment of eyelid swelling is preferably within 200 and 2000 mOsm/Kg, preferably 250 mOsm/Kg-1500 mOsm/Kg, more preferably 260 mOsm/Kg-1250 mOsm/Kg, more preferably 265 mOsm/Kg to 1200 mOsm/Kg and more preferably 400 mOsm/Kg to 1150 mOsm/Kg and more preferably 500 mOsm/Kg to 1100 mOsm/Kg.

In certain embodiments, the topical formulations additionally comprise a preservative. A preservative may typically be selected from a quaternary ammonium compound such as benzalkonium chloride (N-benzyl-N—(C₈-C₁₈ alkyl)-N,N-dimethylammonium chloride), benzoxonium chloride or the like. Examples of preservatives different from quaternary ammonium salts are alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal® II or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In another embodiment, the topical formulations of this invention do not include a preservative. Such formulations would be useful for patients who wear contact lenses, or those who use several topical ophthalmic drops and/or those with an already compromised ocular surface (e.g. dry eye) wherein limiting exposure to a preservative may be more desirable.

The topical formulation may additionally require the presence of a solubilizer, in particular if the active or the inactive ingredients tends to form a suspension or an emulsion. A solubilizer suitable for an above concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and is generally in the range of from approximately 0.0001 to approximately 90% by weight.

Other compounds may also be added to the formulations of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

### 4. Methods of Use

The invention features methods of treating and preventing eyelid swelling in a subject comprising use of the novel formulations described above. For example, a method of treating eyelid swelling comprises administering to the eye surface of the subject a pharmaceutical composition comprising an effective amount of an osmotically active agent and/or vasoconstrictor and/or astringent in a pharmaceutically acceptable carrier. As another example, a method of treating eyelid swelling may comprise administering to the outer and/or inner eyelid surface or ocular surface of the subject a pharmaceutical composition comprising an effective amount of an osmotically active agent and/or vasoconstrictor and/or astringent in a pharmaceutically acceptable carrier. In a particular embodiment, the method of treating eyelid swelling may comprise administering to the outer and/or inner eyelid surface or ocular surface of the subject a pharmaceutical composition comprising a combination of an effective amount of an osmotically active agent and a vasoconstrictor. Various embodiments of such formulations that are suitable for use in the methods of the invention are described above.

In various embodiments, the composition may be administered in the form of an emulsion or suspension, liposome, lotion, ointment, cream, gel, salve, or powder, and sustained or slow release, as well as eyelid lotions, or other types of solid or semi-solid compositions, including formulations described in U.S. Pat. No. 6,806,364. It may also be used as an eye wash or rinse to irrigate the eye. The composition may also be administered in a sprayable form.

The effective amount of osmotically active agent and/or vasoconstrictor and/or astringent in the formulation will depend on absorption, inactivation, and excretion rates of the drug and the delivery rate of the compound from the formulation. In certain embodiments comprising an osmotically active agent, the effective amount will also depend on the concentration of agent required to make the formulation a hyperosmotic solution.

The present invention provides a target osmolarity and/or osmolality range for an ophthalmic composition for treating eyelid swelling. The skilled artisan would readily recognize that if the concentration of the composition is very low, such as the concentrations of the composition of the invention, then the terms osmolarity and osmolality are essentially equivalent and have been used interchangeably herein as applied to the compositions of the invention. Through comfort testing, it was discovered that ophthalmic solutions should have an osmolarity and/or osmolality ranging from less than 2000 mOsm/Kg, and more preferably less than 1050 mOsm/Kg to have acceptable, i.e., tolerable comfort profiles. Therefore, the target osmolality range for a drop formulated for the treatment of eyelid swelling is preferably within 200 and 2000 mOsm/Kg, preferably 250 mOsm/Kg-1500 mOsm/Kg, more preferably 260 mOsm/Kg-1250 mOsm/Kg, more preferably 265 mOsm/Kg to 1200 mOsm/Kg and more preferably 400 mOsm/Kg to 1150 mOsm/Kg and more preferably 500 mOsm/Kg to 1100 mOsm/Kg.

It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of any compound of the present invention will vary depending on the symptoms, age and other physical characteristics of the patient, the nature and severity of the disorder to be treated or prevented, the degree of comfort desired, the route of administration, and the form of the supplement. Any of the subject formulations may be administered in a single dose or in divided doses. Dosages for the formulations of the present invention may be readily determined by techniques known to those of skill in the art or as taught herein.

An effective dose or amount, and any possible effects on the timing of administration of the formulation, may need to be identified for any particular formulation of the present invention. This may be accomplished by routine experiment as described herein. The effectiveness of any formulation and method of treatment or prevention may be assessed by administering the formulation and assessing the effect of the administration by measuring one or more indices associated with the efficacy of the agent and with the degree of comfort to the patient, as described herein, and comparing the post-treatment values of these indices to the values of the same indices prior to treatment or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

The combined use of several agents formulated into the compositions of the present invention may reduce the required dosage for any individual component because the onset and duration of effect of the different components may be complimentary. In such combined therapy, the different agents may be delivered together or separately, and simultaneously or at different times within the day.

Efficacy of the formulations and compositions of the invention in treating and preventing eyelid swelling may be assessed by measuring changes in eyelid swelling, using various methods, including but not limited to ruler measurements, subjective scales (for example, but not limited to, subjective clinical scales that determine swelling as mild, moderate, severe, or 0, 1, 2, or 3, or other appropriate scale), and scanning technology. In a preferred embodiment, changes in eyelid swelling are assessed using 3D scanning technology. Use of 3D scanning technology enables the quantification of the daily fluctuation in lid swelling, which has not been accurately measured previously, to assess the reduction of lid swelling using various formulations of the invention.

### 5. Packaging

The formulations of the present invention may be packaged as either a single dose product or a multi-dose product. The single dose product is sterile prior to opening of the package and all of the composition in the package is intended to be consumed in a single application to one or both eyes of a patient. The use of an antimicrobial preservative to maintain the sterility of the composition after the package is opened is generally unnecessary.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”), other publications by the Food and Drug Administration, and corresponding publications in other countries. Detailed descriptions of the specifications for preservation of ophthalmic pharmaceutical products against microbial contamination and the procedures for evaluating the preservative efficacy of specific formulations are provided in those publications. In the United States, preservative efficacy standards are generally referred to as the “USP PET” requirements. (The acronym “PET” stands for “preservative efficacy testing.”)

The use of a single dose packaging arrangement eliminates the need for an antimicrobial preservative in the compositions, which is a significant advantage from a medical perspective, because conventional antimicrobial agents utilized to preserve ophthalmic compositions (e.g., benzalkonium chloride) may cause ocular irritation, particularly in patients suffering from dry eye conditions or pre-existing ocular irritation. However, the single dose packaging arrangements currently available, such as small volume plastic vials prepared by means of a process known as “form, fill and seal”, have several disadvantages for manufacturers and consumers. The principal disadvantages of the single dose packaging systems are the much larger quantities of packaging materials required, which is both wasteful and costly, and the inconvenience for the consumer. Also, there is a risk that consumers will not discard the single dose containers following application of one or two drops to the eyes, as they are instructed to do, but instead will save the opened container and any composition remaining therein for later use. This improper use of single dose products creates a risk of microbial contamination of the single dose product and an associated risk of ocular infection if a contaminated composition is applied to the eyes.

While the formulations of this invention are preferably formulated as “ready for use” aqueous solutions, alternative formulations are contemplated within the scope of this invention. Thus, for example, the active ingredients, surfactants, salts, chelating agents, or other components of the ophthalmic solution, or mixtures thereof, can be lyophilized or otherwise provided as a dried powder or tablet ready for dissolution (e.g., in deionized, or distilled) water. Because of the self-preserving nature of the solution, sterile water is not required.

### 6. Kits

In still another embodiment, this invention provides kits for the packaging and/or storage and/or use of the formulations described herein, as well as kits for the practice of the methods described herein. Thus, for example, kits may comprise one or more containers containing one or more ophthalmic preparations, tablets, or capsules of this invention. The kits can be designed to facilitate one or more aspects of shipping, use, and storage.

The kits may optionally include instructional materials containing directions (i.e., protocols) disclosing means of use of the formulations provided therein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g. CD ROM), and the like). Such media may include addresses to interne sites that provide such instructional materials.

### EXAMPLES

The invention, having been generally described, may be more readily understood by reference to the following examples, which are included merely for purposes of illustration of certain aspects and embodiments of the present invention, and are not intended to limit the invention in any way.

### Example 1

### Use of Naphazoline 0.1% Ophthalmic Solution as a Treatment for Morning Eyelid Edema

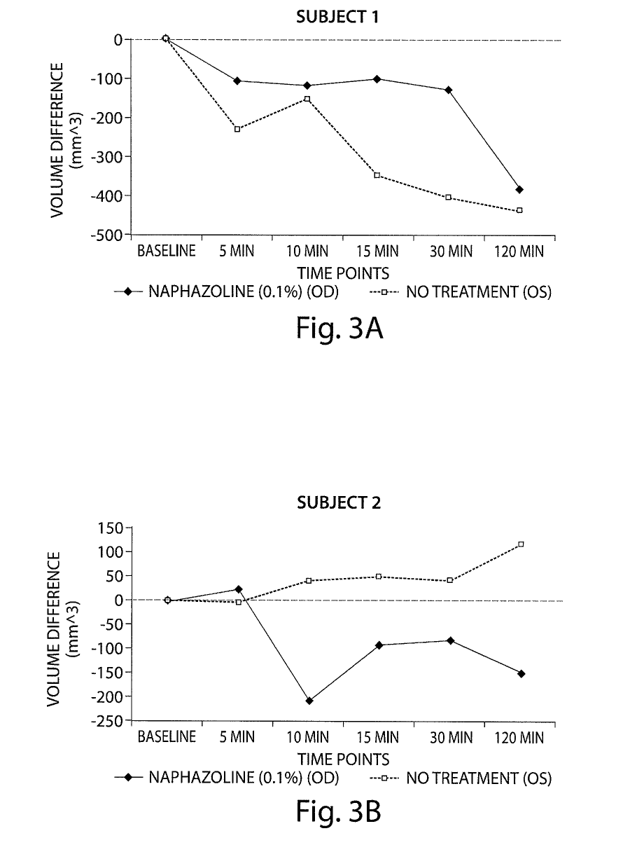

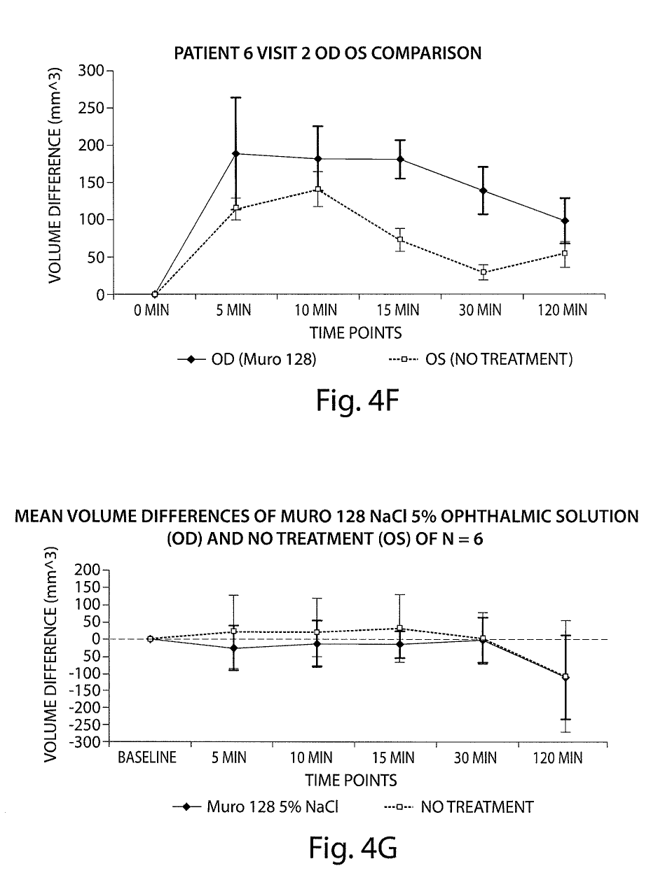

In this study, the efficacy of naphazoline 0.1% ophthalmic solution was evaluated for treatment for eyelid edema. The eyelid volume for 11 subjects participating in the study was recorded in the afternoon of Day 1 and again upon arrival to the clinic the following morning (Day 2). All subjects showed an increase in eyelid swelling at the morning scan during Visit 2. The natural progression of morning eyelid swelling, as measured using 3D scanning technology is depicted in FIG. 12. The increase ranged from 14 mm³ to 659 mm³. Subjects were then dosed with naphazoline 0.1% (vasoconstrictor) in the right eye and eyelid volume was assessed at 5, 10, 15, 30, and 120 minutes following treatment using a 3D scanner. As shown in FIG. 3, two drops of naphazoline 0.1% solution caused a reduction of eyelid swelling in most subjects. Nine out of 11 subjects showed greater decrease in volume in the treatment eye (naphazoline 0.1%) than the non-treatment eye up to the 30 minute timepoint (FIGS. 3B-E, G-K). Up to the 120 minute timepoint, 7 out of 11 subjects showed greater decrease in volume in the right eye than the left eye (FIGS. 3B, C, E, G-I, K). A summary of all the patient data is depicted if FIG. 3L.

Overall, these results demonstrated efficacy of the naphazoline 0.1% for ability to reduce eyelid swelling in patients with morning lid swelling and not in a diseased eye or eye with current vasodilation where a vasoconstrictor would be typically used.

### Example 2

### Use of a Colloidal Osmotic Agent, NaCl 5% Ophthalmic Solution, for Treatment of Morning Eyelid Edema

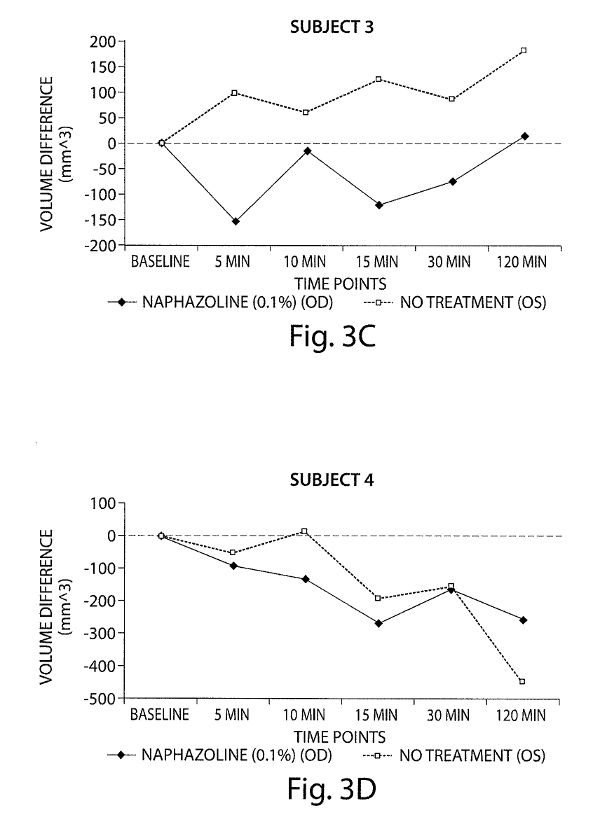

In a preliminary study with a similar design to that described above, NaCl 5% ophthalmic solution was evaluated as a potential treatment for eyelid edema. Two drops of medication were applied topically and caused a reduction of eyelid swelling in several patients (FIG. 4). Eyelid swelling was assessed using a 3D scanner at 5, 10, 15, 20, 30, and 120 minutes post-treatment. Three patients demonstrated a reduction in eyelid swelling through 15 minutes post-instillation. In one subject, this reduction was pronounced and was present through the 120 minute assessment time point. In the remaining three patients, treatment was not effective.

Overall, these results demonstrated some efficacy of the NaCl 5% for ability to reduce eyelid swelling in certain patients. An assessment of mean change from baseline (FIG. 4G) suggests that NaCl treatments were numerically superior to negative controls, though the differences were not statistically significant in this small study.

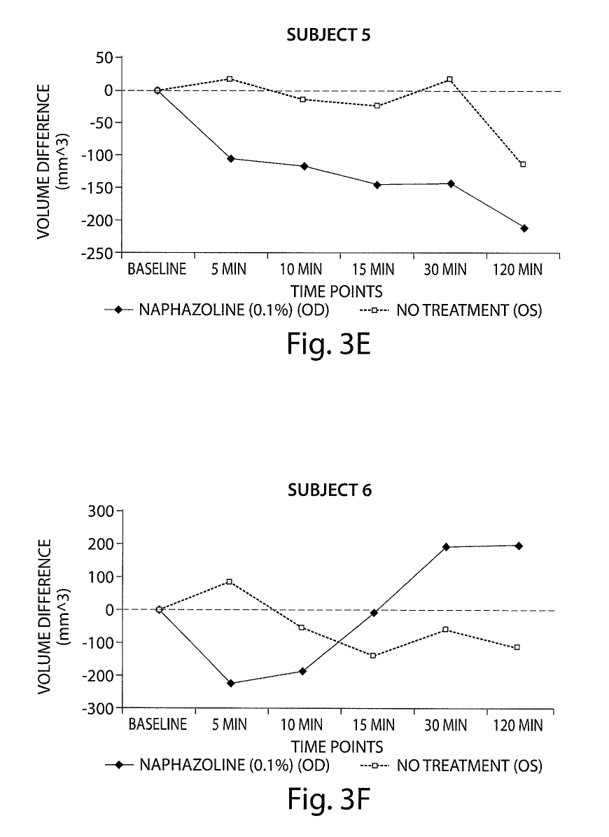

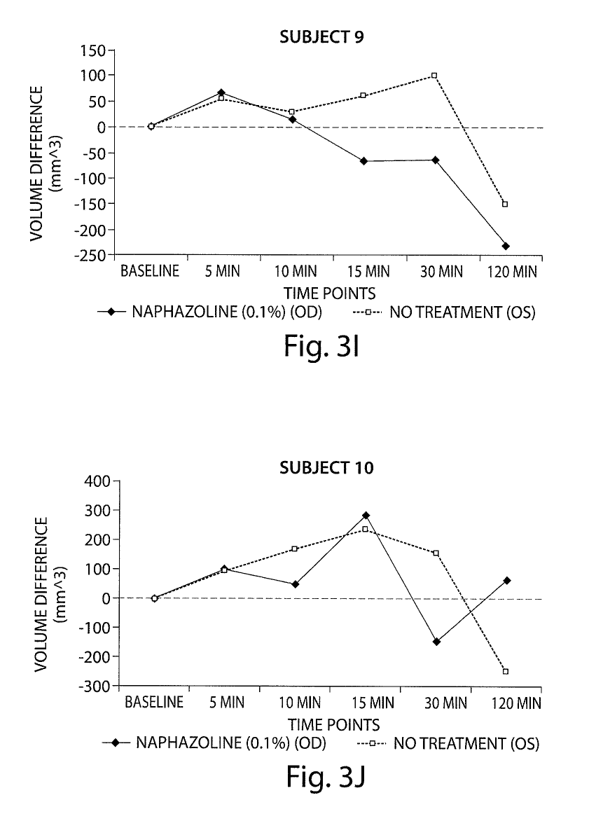

Further, naphazoline 0.1% in combination with NaCl 5% demonstrates superior efficacy in reducing eyelid swelling in patients as compared to the individual components naphazoline 0.1% alone and NaCl 5% alone (FIGS. 5-7).

### Example 3

### Use of Naphazoline 0.05%/NaCl 5% Ointment for Treatment of Morning Eyelid Edema

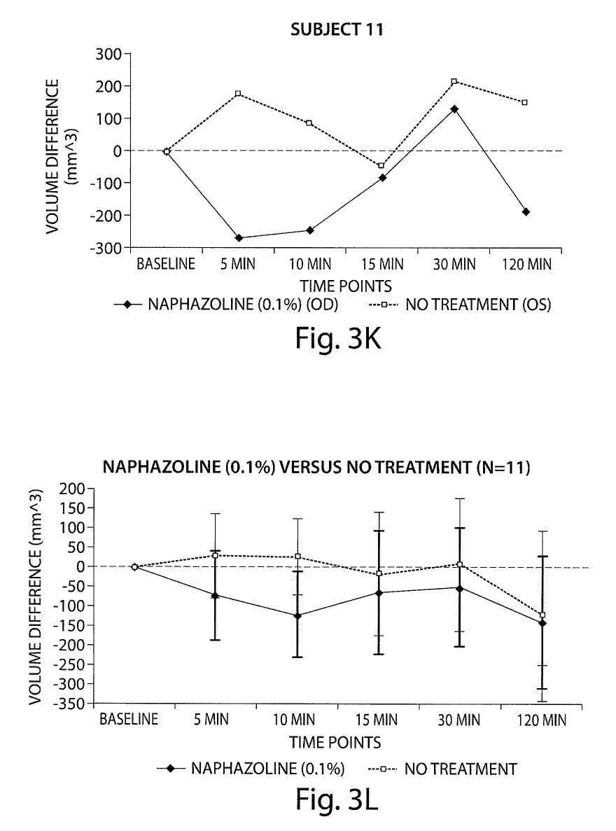

The efficacy of naphazoline hydrochloride 0.05% dissolved in NaCl 5% ophthalmic ointment in preventing morning eyelid swelling was evaluated in four (4) patients. Three dimensional scans were taken of each patient and each eye during the afternoon between 4:30 to 5:30 pm. Each patient was asked to take home a vial containing NaCl 5% ophthalmic ointment containing naphazoline hydrochloride 0.05% and apply the ointment into the conjunctival sac of the right eye immediately prior to sleep. The following morning, between 7:30 to 8:00 am, patients were scanned again for each eye. The mean volumes of the upper and lower eyelid regions were calculated for both afternoon and morning scans of each patient. The differences between the means were also calculated. Results showed that the treatment eye had approximately half the swelling of the untreated eye (FIG. 8).

The final formulation used in this study was: sodium chloride 5% in lanolin, mineral oil, purified water, white petrolatum, and naphazoline hydrochloride 0.05%.

### Example 4

### Use of Sodium Chloride (2.5%)/Naphazoline (0.1%) and Sodium Chloride (5%)/Naphazoline (0.1%) for the Treatment of Morning Eyelid Edema

The efficacy of naphazoline 0.1% in combination with sodium chloride 2.5% solution in treating and/or preventing morning eyelid swelling was evaluated, as measured by 3D scanning technology.

Sodium chloride 2.5% was formulated with water. Naphazoline was then dissolved in NaCl 2.5% solution to formulate naphazoline 0.1% concentration.

A total of 6 subjects (male, between the ages of 25 and 29) were evaluated. At the start of the study, five (5) baseline scans were performed per subject and eye using a 3D scanner. The next day, subjects were asked to five (5) scans of each eye, identical to Visit 1, were then taken.

Subjects received the 2 drops (40 μl each) of the combination treatment, with one minute apart each drop in one eye and no treatment in the other eye. Five (5) scans of each eye were taken, identical to Visit 1, 20 minutes after second drop instillation. Subjects were asked to subjectively grade their eyelid swelling post treatment based on a comfort scale of 0 to 10 (0 indicating most comfortable, 10 indicating least comfortable). Digital photos were also taken at baseline and at 20 minutes post treatment.

The mean comfort level immediately after instillation was 3.3. Mean eyelid volume increase in the morning was 243 and 309 mm³ for the right eye and left eye, respectively. The mean decrease 20 minutes after treatment was −100 and −14 mm³ for the treatment eye and no treatment eye, respectively.

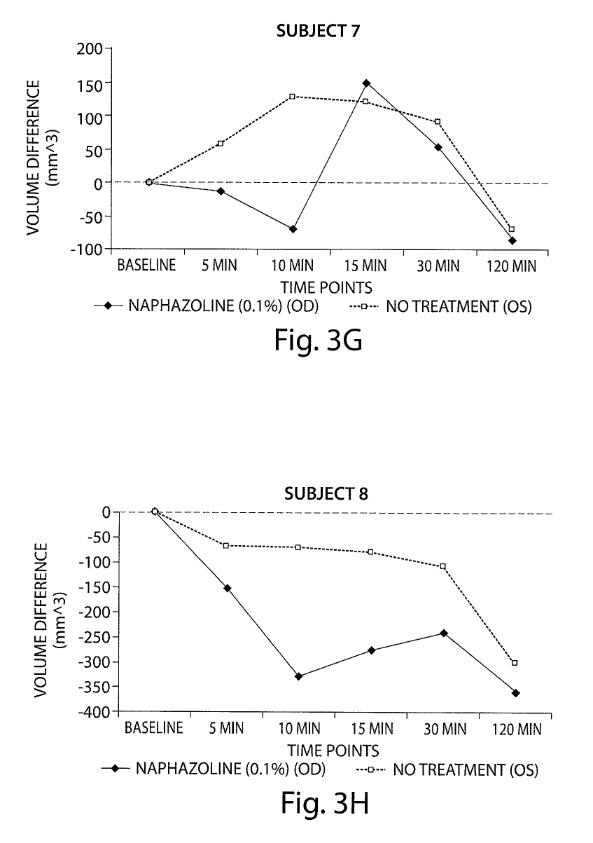

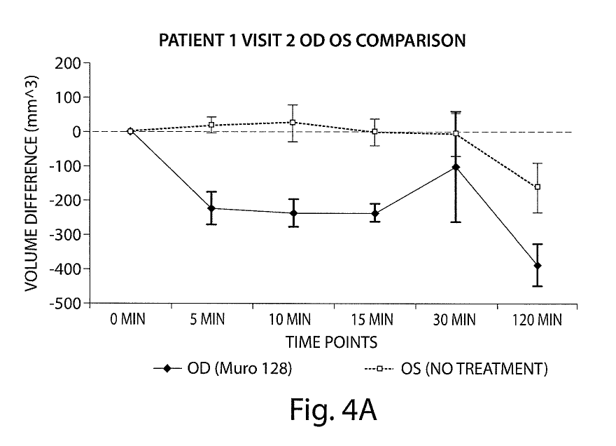

These results suggest that the NaCl 2.5% in combination with naphazoline 0.1% did reduce morning eyelid swelling (FIG. 9). In comparison with NaCl 5% in combination with naphazoline 0.1%, NaCl 2.5% was less efficacious (approximately by half) (See FIGS. 6 and 9). This suggests that the efficacy of NaCl in treating morning eyelid swelling is directly related to concentration.

In terms of comfort level, the NaCl 2.5% was more comfortable than the NaCl 5%, which is an improvement. The mean comfort level for this study (3.3) was more comfortable than the NaCl 5%/naphazoline 0.1% combination (5.8).

### Example 5

### Use of Sucrose 50% and Naphazoline (0.1%) for the Treatment of Morning Eyelid Edema

The efficacy of naphazoline (0.1%) in combination with sucrose 50% solution in treating and/or preventing morning eyelid swelling was evaluated, as measured by 3D scanning technology.

Sucrose was formulated with water to yield a 50% concentration. Naphazoline was then dissolved in the sucrose solution to formulate naphazoline (0.1%) concentration.

A total of 6 subjects were evaluated and methods were similar to previous experiments. At the start of the study, five (5) baseline scans were performed per subject and eye using a 3D scanner. The next day, subjects were asked to five (5) scans of each eye, identical to Visit 1, were then taken.

Subjects received the 2 drops (40 μl each) of the combination treatment, with one minute apart each drop in one eye and no treatment in the other eye. Five (5) scans of each eye were taken, identical to Visit 1, 20 minutes after second drop instillation. Subjects were asked to subjectively grade their eyelid swelling post treatment based on a comfort scale of 0 to 10 (0 indicating most comfortable, 10 indicating least comfortable). Digital photos were also taken at baseline and at 20 minutes post treatment.

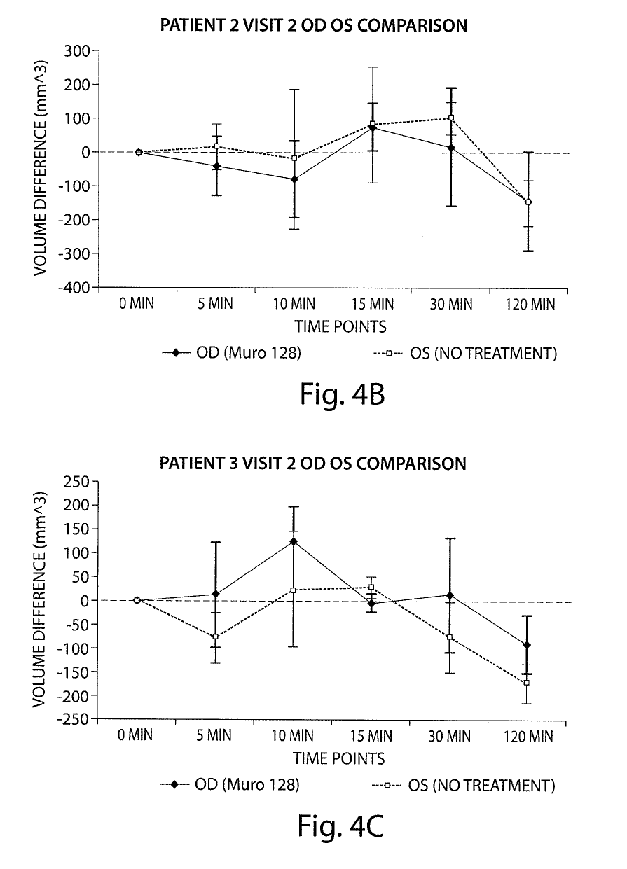

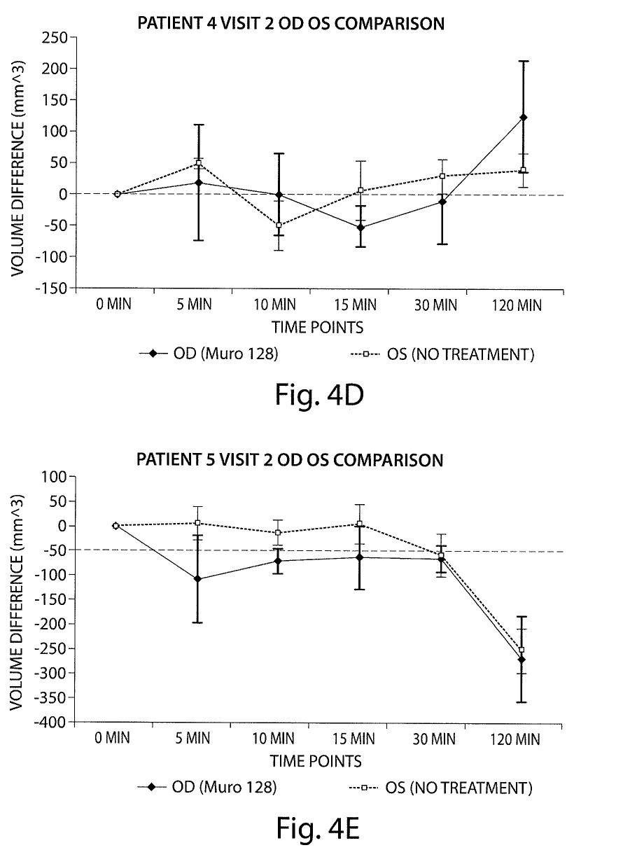

These results suggest that the sucrose 50%/naphazoline 0.1% formulation did reduce morning eyelid swelling (FIG. 10). Further, sucrose 50% in combination with naphazoline 0.1% demonstrates superior efficacy in reducing eyelid swelling in patients as compared to the individual components sucrose 50% alone and naphazoline 0.1% alone (FIGS. 11A and 11B). Mean comfort of the study group was 5.2.

### Example 6

### Use of Topical Phenylephrine 0.25% Ointment for the Treatment of Morning Eyelid Edema

The efficacy of topical phenylephrine 0.25% ointment treating and/or preventing morning eyelid swelling was evaluated, as measured by 3D scanning technology.

A total of 6 subjects were evaluated and methods were similar to previous experiments. At the start of the study, five (5) baseline scans were performed per subject and eye using a 3D scanner. The next day, subjects were asked to five (5) scans of each eye, identical to Visit 1, were then taken.

Subjects received the 2 drops (40 μl each) of the combination treatment, with one minute apart each drop in one eye and no treatment in the other eye. Five (5) scans of each eye were taken, identical to Visit 1, 20 minutes after second drop instillation. Subjects were asked to grade their eyelid swelling post treatment. Digital photos were also taken at baseline and at 20 minutes post treatment.

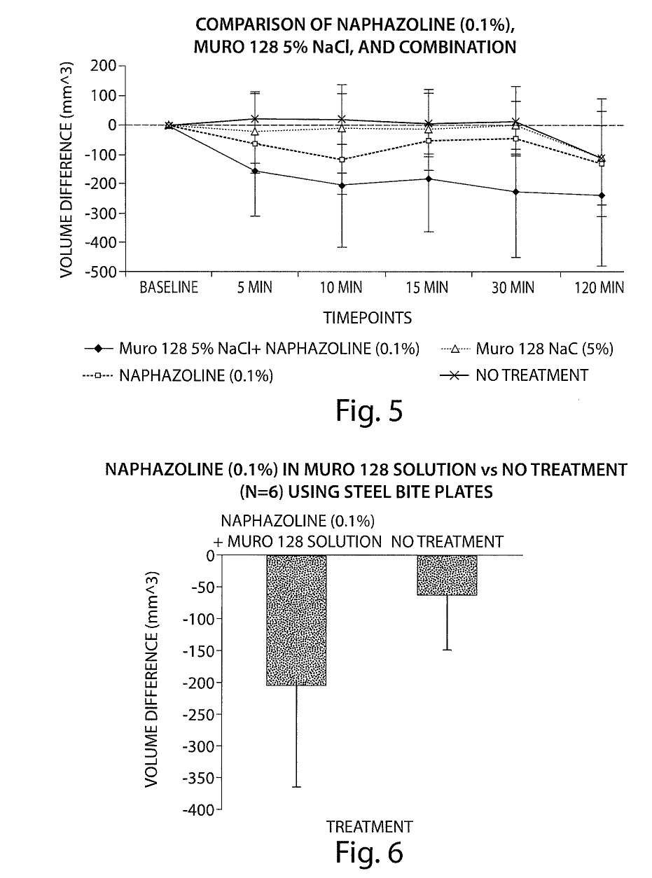

These results suggest that phenylephrine 0.25% ointment applied topically on the lower eyelid did reduce morning eyelid swelling (FIG. 13).

### Example 7

### Use of Mannitol 12.5% with Naphazoline 0.1% and NaCl 5% Solution for the Treatment of Morning Eyelid Edema

The efficacy of mannitol 12.5% with naphazoline 0.1% in NaCl 5% solution for treating and/or preventing morning eyelid swelling was evaluated, as measured by 3D scanning technology.

A total of 6 subjects were evaluated and methods were similar to previous experiments. At the start of the study, five (5) baseline scans were performed per subject and eye using a 3D scanner. The next day, subjects were asked to five (5) scans of each eye, identical to Visit 1, were then taken.

Subjects received the 2 drops (40 μl each) of the combination treatment, with one minute apart each drop in one eye and no treatment in the other eye. Five (5) scans of each eye were taken, identical to Visit 1, 20 minutes after second drop instillation. Subjects were asked to grade their eyelid swelling post treatment. Digital photos were also taken at baseline and at 20 minutes post treatment.

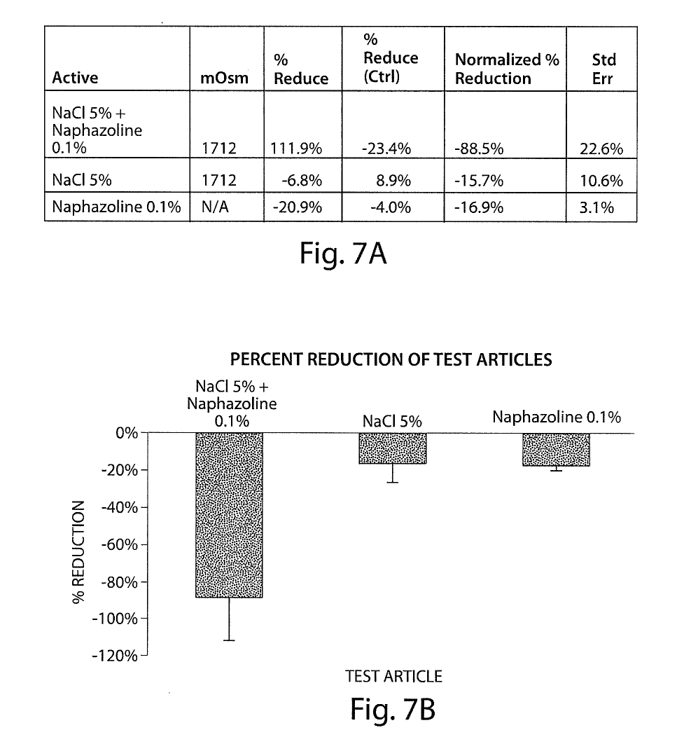

These results suggest that the mannitol/naphazoline/NaCl combination did reduce morning eyelid swelling (FIG. 14).

### Example 8

### Use of Mannitol 12.5% Solution for the Treatment of Morning Eyelid Edema

The efficacy of mannitol 12.5% solution for treating and/or preventing morning eyelid swelling was evaluated, as measured by 3D scanning technology.

A total of 6 subjects were evaluated and methods were similar to previous experiments. At the start of the study, five (5) baseline scans were performed per subject and eye using a 3D scanner. The next day, subjects were asked to five (5) scans of each eye, identical to Visit 1, were then taken.

Subjects received the 2 drops (40 μl each) of the combination treatment, with one minute apart each drop in one eye and no treatment in the other eye. Five (5) scans of each eye were taken, identical to Visit 1, 20 minutes after second drop instillation. Subjects were asked to grade their eyelid swelling post treatment. Digital photos were also taken at baseline and at 20 minutes post treatment.

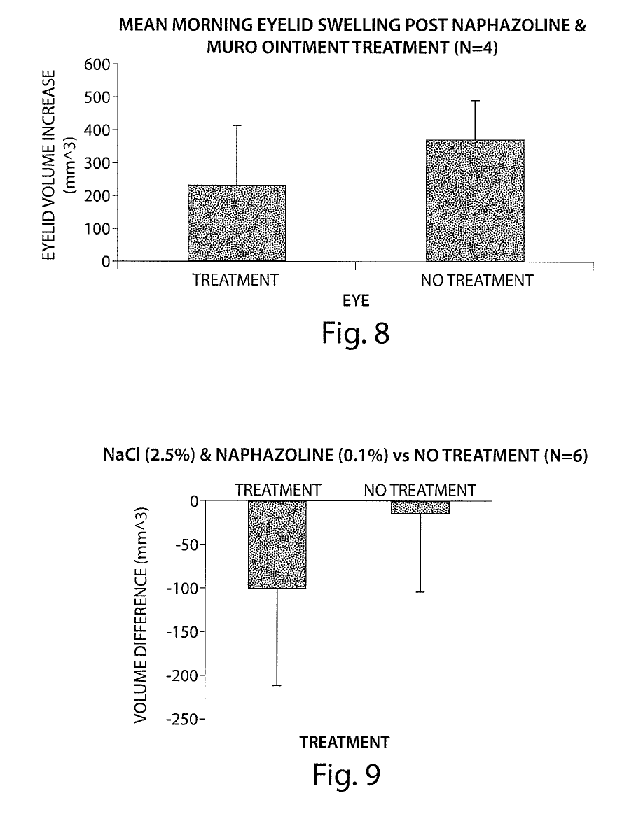

These results suggest that the mannitol 12.5% solution did reduce morning eyelid swelling (FIG. 15).

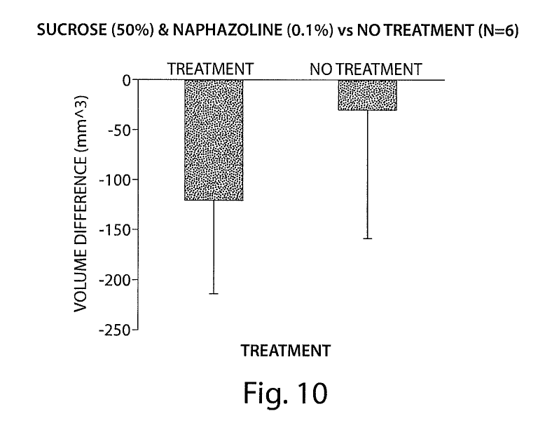

FIGS. 16A and 16B summarize the results of the studies described in Examples 1-8, and depict the superior efficacy of the combined formulations described in Examples 1-8 as compared to the individual agents used alone for the treatment of morning eyelid swelling. These results show that the combined formulations as described above were each efficacious in reducing eyelid swelling, and in most instances, the combined formulations provided a synergistic effect as compared to the individual agents alone.

### Example 9

### Use of Sucrose 50% Solution for the Treatment of Morning Eyelid Edema

The efficacy of Sucrose (50%) for treating and/or preventing morning eyelid swelling was evaluated, as measured by 3D scanning technology.

A total of 6 subjects were evaluated and methods were similar to previous experiments. At the start of the study, five (5) baseline scans were performed per subject and eye using a 3D scanner. The next day, subjects were asked to five (5) scans of each eye, identical to Visit 1, were then taken.

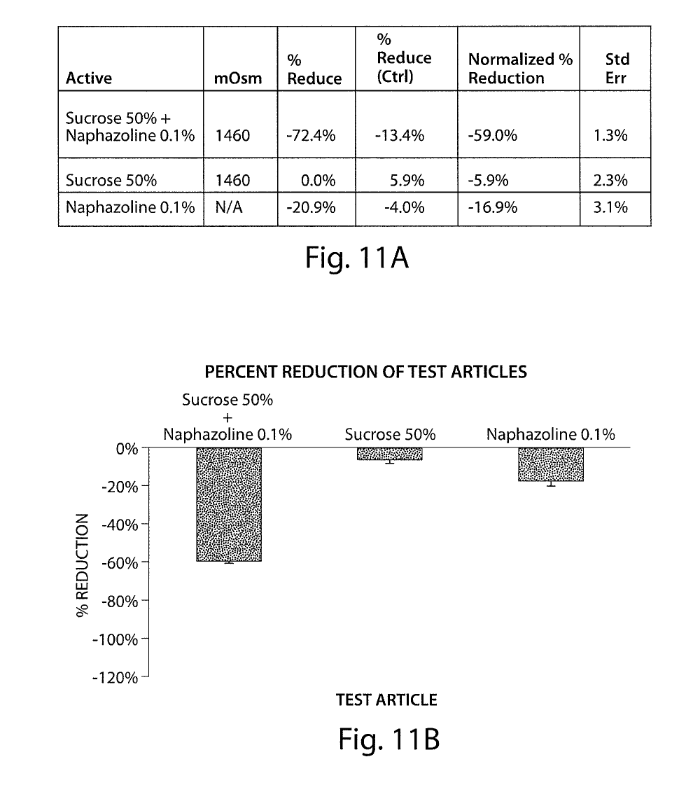

Subjects received the 2 drops (40 μl each) of the combination treatment, with one minute apart each drop in one eye and no treatment in the other eye. Five (5) scans of each eye were taken, identical to Visit 1, 20 minutes after second drop instillation. Subjects were also asked to subjectively grade their eyelid swelling post treatment based on a comfort scale of 0 to 10 (0 indicating most comfortable, 10 indicating least comfortable). These results suggest that the sucrose 50% solution minimally reduced morning eyelid swelling (FIG. 17). The mean comfort of the study group was 4.5.

### Example 10

### Osmolality and Comfort

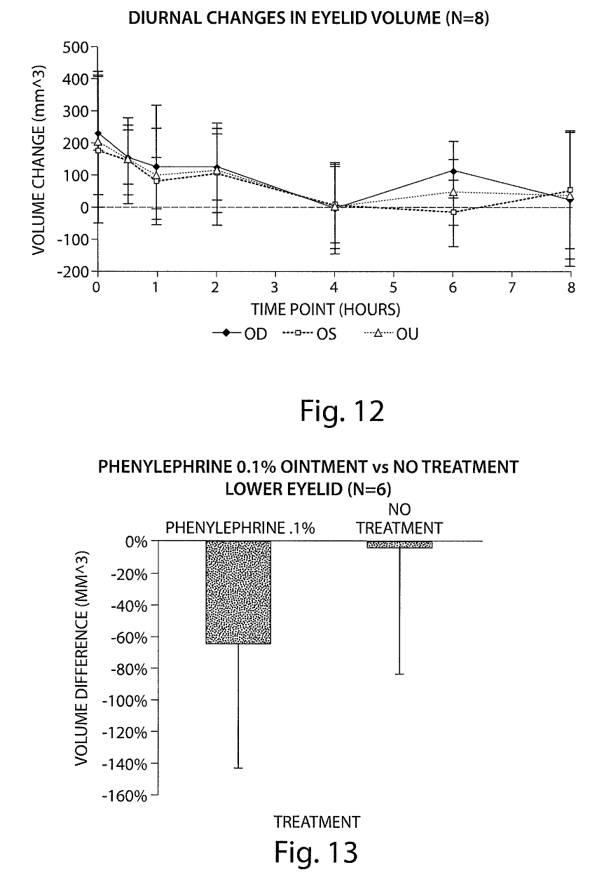

A correlation between osmolality of the test article and comfort was evaluated to determine a relationship, if any. The table shown in FIG. 18A depicts the corresponding osmolality value, and the mean comfort level immediately after instillation of each test article (based on a subjective scale of 0 to 10, 0 indicating most comfortable, 10 indicating least comfortable).

Without intending to be bound by any theory, these results suggest a direct relationship between osmolality and comfort, where higher osmolality induces greater discomfort (FIG. 18B). Based on this data, it is hypothesized that the maximum and ideal osmolality of a test article to reduce morning eyelid swelling without inducing high discomfort ranges from less than 2000 mOsm/Kg, and preferably is between within 200 mOsm/Kg to 2000 mOsm/Kg, more preferably 250 mOsm/Kg to 1500 mOsm/Kg, more preferably 260 mOsm/Kg to 1250 mOsm/Kg, even more preferably 265 mOsm/Kg to 1200 mOsm/Kg.

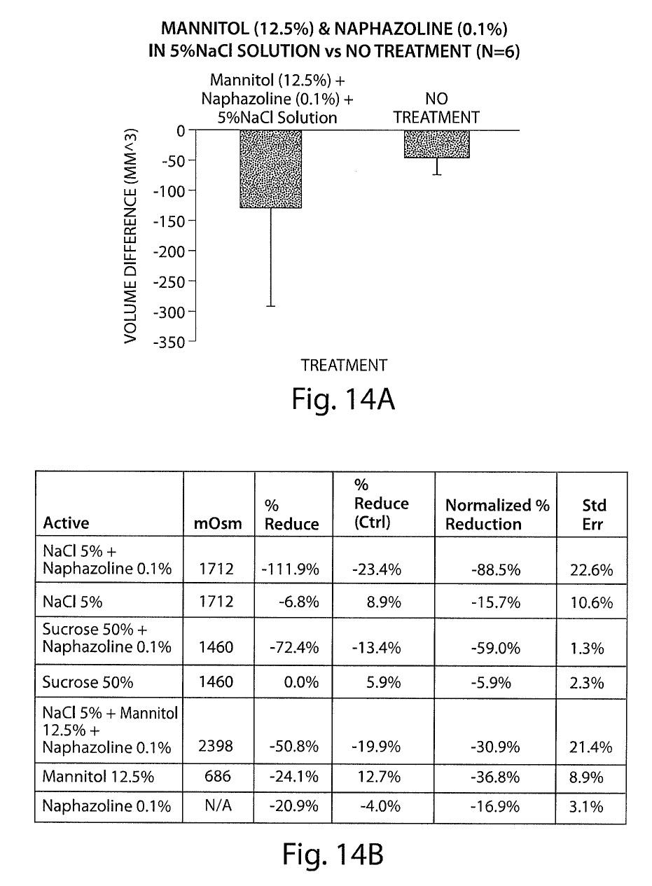

FIG. 19 shows comfort data on additional ophthalmic formulations containing combinations of naphazoline (0.1% and 0.09%) and NaCl 3%; oxymetazoline (0.03%, 0.04%, 0.05%) and mannitol (12.5%, 6%, and 3%); oxymetazoline 0.05%, mannitol 6% and NaCl 3%; oxymetazoline 0.05% and NaCl 3%; and oxymetazoline 0.05% and glycerol 7.5%; and oxymetazoline 0.05% alone. Comfort level was measured immediately after instillation of each test article, based on the 0-10 subjective scale as previously described. The osmolality of each of these formulations is predicted to be within the targeted range for an acceptable comfort profile (i.e., within 500 mOsm/Kg to 1100 mOsm/Kg).

### Example 11

### Use of Naphazoline (0.09%)/Sodium Chloride (3%) for the Treatment of Morning Eyelid Edema

A single center, double-masked randomized, contralateral, placebo controlled study was designed to assess the pattern of morning eyelid swelling upon awakening in a hotel setting and at home daily for 6 days, and to assess the efficacy of a single dose of naphazoline 0.09%/NaCl 3% ophthalmic solution compared to placebo, in the reduction of morning eyelid swelling.

The naphazoline 0.09%/NaCl 3% ophthalmic solution was prepared as shown in Table 1:

TABLE 1
Naphazoline 0.09%/NaCl 3% ophthalmic solution

| Target | Quantity | |
|---|---|---|
| Quantity (%) | (mg/mL) | Raw Material Description |
| 0.09 | 0.9 | Naphazoline hydrochloride, USP |
| 3.0 | 30.0 | Sodium chloride, USP |
| 0.1 | 1.0 | Edetate disodium, USP |
| 0.5 | 5.0 | Boric Acid, NF |
| 0.01 | 0.1 | Benzalkonium chloride, NF |

Sodium hydroxide 0.5N or hydrochloric acid 0.5N was used to adjust the pH to 6.0 and the formulation was QS to 1 mL using purified water (USP).

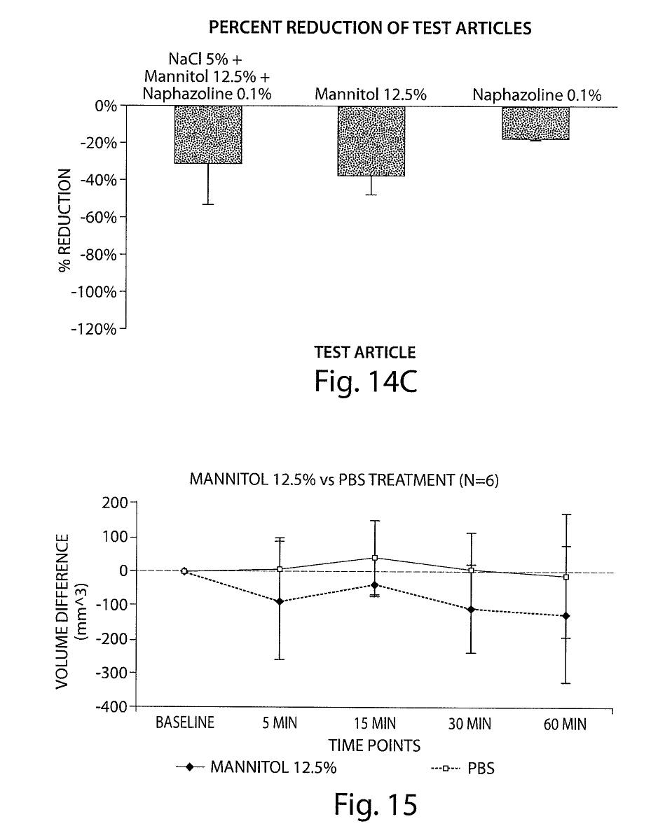

A total of 20 female subjects, mean age 50 years old, were evaluated as follows. Screening and baseline lid swelling assessments were taken in a hotel setting in the evening (visit 1, evening (pm)) and in the following morning upon awakening (visit 2, baseline) then in ten minute intervals for up to one hour using a regional/global lid swelling scale. For subjective assessment of lid swelling, the eyelid and surrounding area was divided into 4 different regions of the ocular region, including the upper and lower eyelids (regions 1 and 2, respectively) and the region immediately above and below the upper and lower eyelids (regions 3 and 4, respectively). Subjects were asked to subjectively score lid swelling in each region on a scale of 0-3. Subjects were also asked to subjectively score lid swelling on a global (i.e. overall) basis. A score of zero (“0”) was used to indicate that the subject did not detect any swelling in the assessed region or globally; a score of “3” was used to indicate that the subject detected definite swelling in the assessed region or globally. The mean scores for each the 4 eyelid regions during the baseline measurements at visits 1 and 2 are shown in FIG. 20. As shown in FIG. 20, the greatest amount of eyelid swelling was detected in Region 3 as well as the globally.

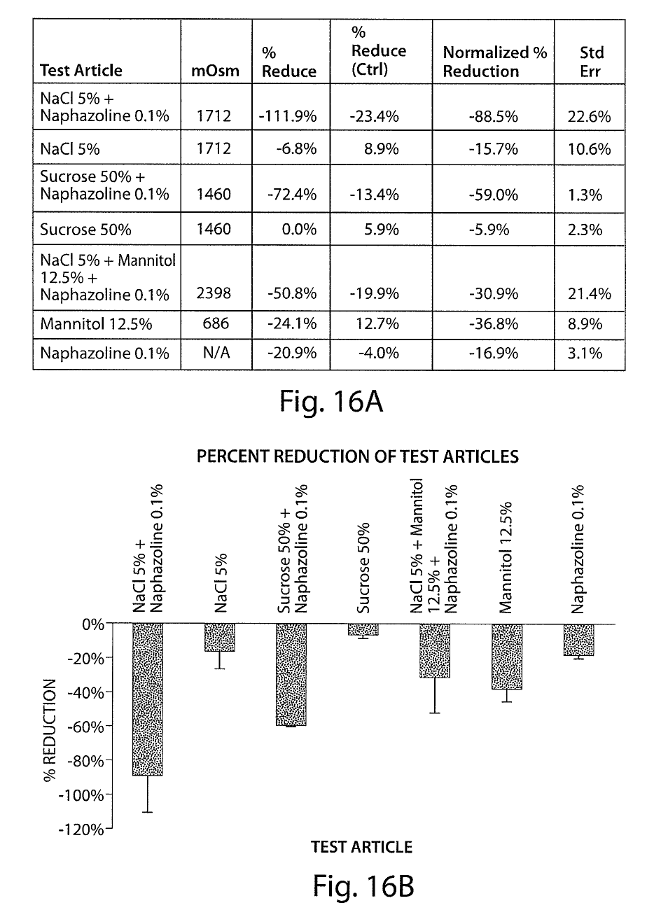

After the baseline assessments at visits 1 and 2 were made, subjects were sent home for a 6 day period with a diary and were asked to subjectively grade and record their eyelid swelling in the morning and evening of each day using the regional/global lid swelling scale. The mean global scores by time of day for the 6 day period is shown in FIG. 21. As shown in FIG. 21, the subjects consistently experienced an increase in eyelid swelling in the morning, as compared to the prior evening over the entire 6 day time period.

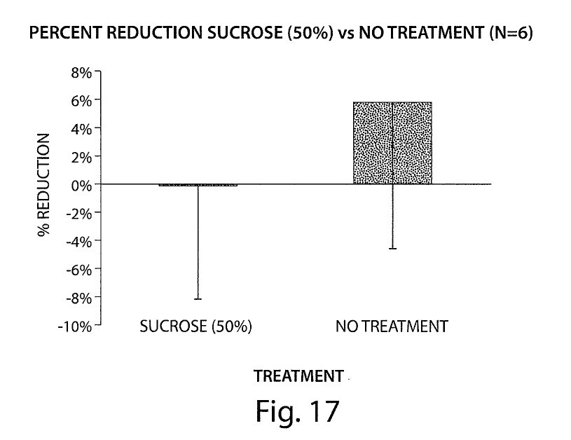

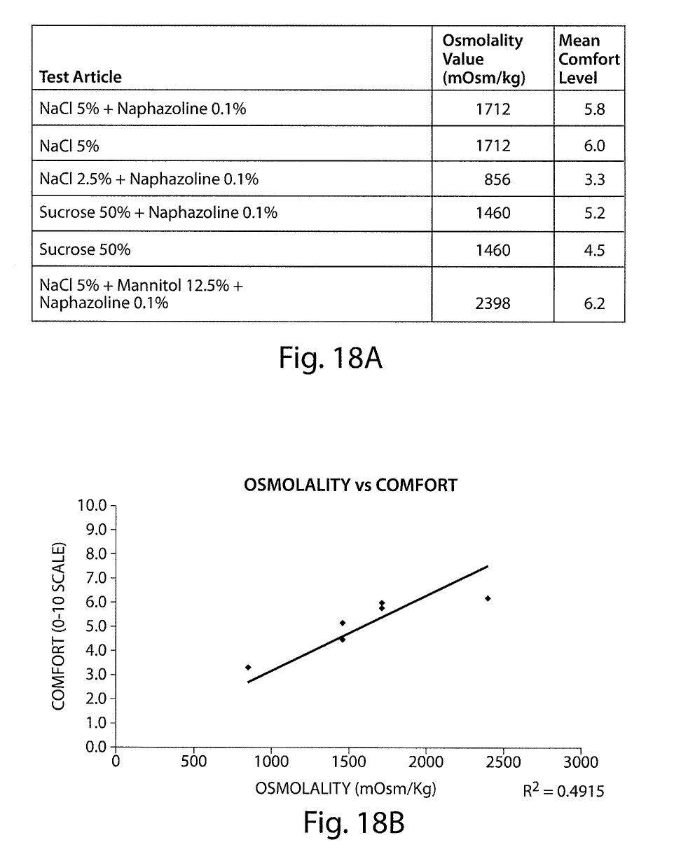

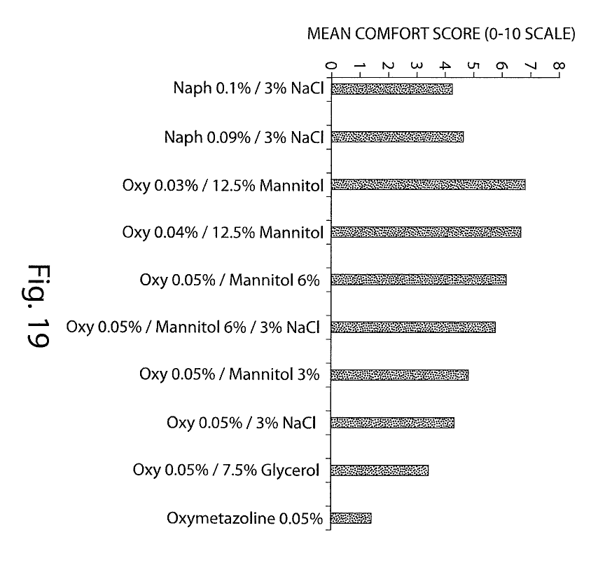

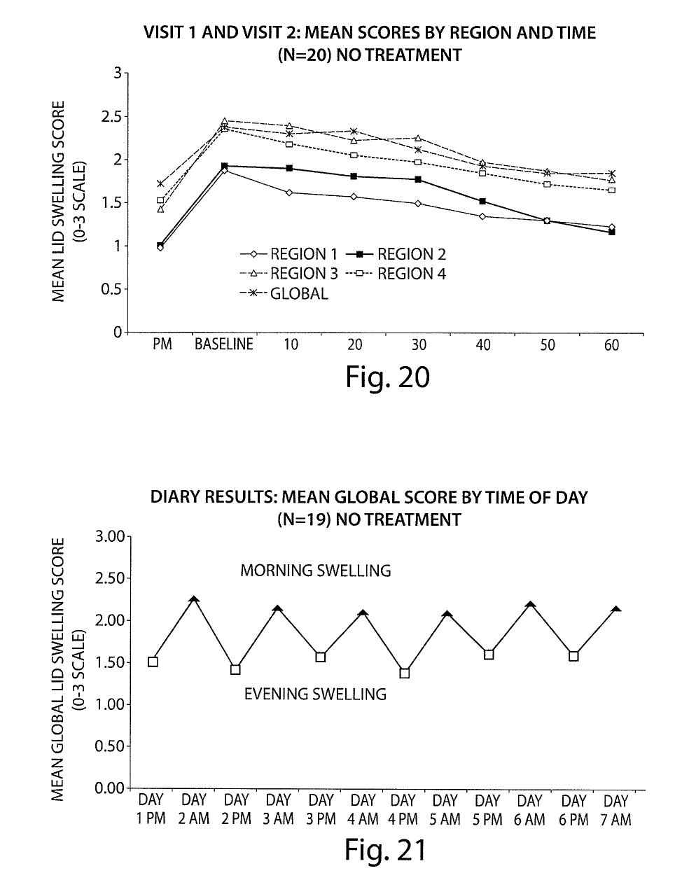

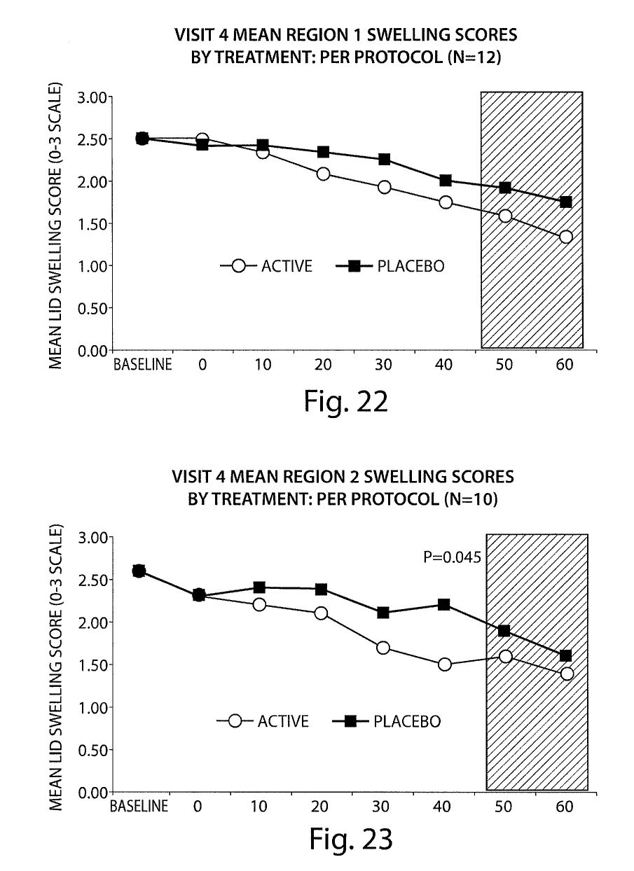

At the end of the 6 day period, subjects returned to the hotel for the treatment arm of the study. Evening (visit 3) and morning (visit 4) subjective lid swelling assessments were again made in the hotel setting using the regional/global lid swelling scale as previously described. Immediately following the morning lid swelling assessment at visit 4, subjects received one drop of naphazoline 0.09%/NaCl 3% in one eye, and Tears Naturale II artificial tears (placebo) in the fellow eye. Subjective post-treatment lid swelling assessments were made in ten minute intervals for 1 hour using the regional/global lid swelling scale, as well as objective assessment of lid swelling improvement (i.e., reduction) using digital photography. For the objective assessment, masked graders were asked to evaluate the digital photos and assess whether lid swelling was better, worse or the same as baseline measurements. The subjective post-treatment lid swelling assessment results are shown in FIGS. 22-26. As shown in FIGS. 22-26, naphazoline 0.09%/NaCl was more effective than placebo in reducing morning eyelid swelling in each of the 4 designated eyelid regions assessed, as well as globally.

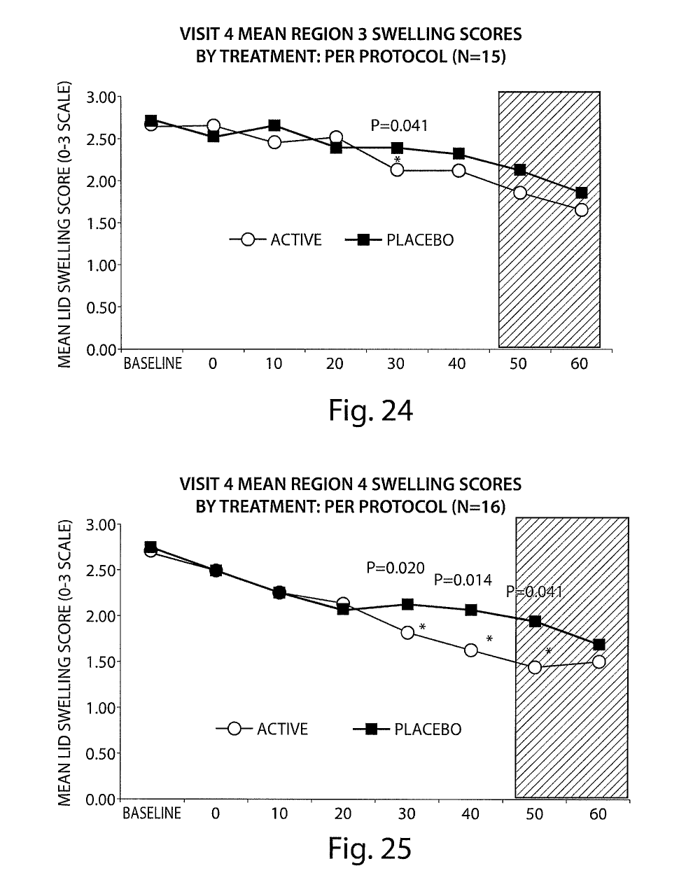

The mean comfort of the treatment was also evaluated. After instillation of naphazoline 0.09%/NaCl 3%, subjects were asked to grade comfort of the drop in their eye on a subjective scale of 0-10 (0 indicating most comfortable, 10 indicating least comfortable). The results are shown in FIG. 27. As shown in FIG. 27, the treatment arm had a 4.0 mean comfort score as compared to placebo, which was more comfortable (mean comfort score 1.5). The osmolality of the naphazoline 0.09%/NaCl 3% ophthalmic solution is predicted to be within the targeted range for an acceptable comfort profile (i.e., within 500 mOsm/Kg to 1100 mOsm/Kg). Only three subjects reported transient stinging post instillation of naphazoline 0.09%/NaCl 3% in the actively treated eye.

In summary, naphazoline 0.09%/NaCl 3% treated eyes had lower eyelid swelling scores across all regions 40 minutes post instillation. All subjects reported the same or better global scores for the active eye as compared to placebo (p-value=0.001). Global treatment effect was 0.4 unit reduction. One dose of naphazoline 0.09%/NaCl 3% was safe and well tolerated, with a mean comfort score of 4.0. Diary data showed a consistent pattern of morning and evening lid swelling across the 6 day time period in between the baseline hotel setting and treatment setting.

### Example 12

### Comparison of Naphazoline (0.09%)/Sodium Chloride (3%), Oxymetazoline 0.05%/NaCl 3%, Naphazoline 0.09%/Glycerol 7.5%, and Oxymetazoline 0.05%/Glycerol 7.5% for the Treatment of Morning Eyelid Edema

A single center, contralateral, study was designed to assess and compare the efficacy of single doses of naphazoline 0.09%/NaCl 3% ophthalmic solution, oxymetazoline 0.05%/NaCl 3% ophthalmic solution, naphazoline 0.09%/Glycerol 7.5% ophthalmic solution, and oxymetazoline 0.05%/Glycerol 7.5% ophthalmic solution, in the reduction of morning eyelid swelling.

The ophthalmic solutions were prepared as indicated in Tables 2-5.

TABLE 2
Naphazoline 0.09%/NaCl 3% ophthalmic solution

| Target | Quantity | |
|---|---|---|
| Quantity (%) | (mg/mL) | Raw Material Description |
| 0.09 | 0.9 | Naphazoline hydrochloride, USP |
| 3.0 | 30.0 | Sodium chloride, USP |
| 0.1 | 1.0 | Edetate disodium, USP |
| 0.5 | 5.0 | Boric Acid, NF |
| 0.01 | 0.1 | Benzalkonium chloride, NF |

*Osmolality: 1030 mOsm/Kg

TABLE 3
Naphazoline 0.09%/Glycerol 7.5% ophthalmic solution

| | Target | |
|---|---|---|
| Target | Quantity | |
| Quantity (%) | (mg/mL) | Raw Material Description |
| 0.09 | 0.9 | Naphazoline hydrochloride, USP |
| 7.5 | 75.0 | Glycerol, USP |
| 0.10 | 1.0 | Edetate disodium, USP |
| 0.5 | 5.0 | Boric Acid, NF |
| 0.01 | 0.1 | Benzalkonium chloride, NF |

*Osmolality: 938 mOsm/kg

TABLE 4
Oxymetazoline 0.05%/NaCl 3% ophthalmic solution

| Target | Quantity | |
|---|---|---|
| Quantity (%) | (mg/mL) | Raw Material Description |
| 0.05 | 0.5 | Oxymetazoline hydrochloride, USP |
| 3.0 | 30.0 | Sodium chloride, USP |
| 0.1 | 1.0 | Edetate disodium, USP |
| 0.5 | 5.0 | Boric Acid, NF |
| 0.01 | 0.1 | Benzalkonium chloride, NF |

*Osmolality: 1027 mOsm/Kg

TABLE 5
Oxymetazoline 0.05%/Glycerol 7.5% ophthalmic solution

| Target | Quantity | |
|---|---|---|
| Quantity (%) | (mg/mL) | Raw Material Description |
| 0.05 | 0.5 | Oxymetazoline hydrochloride, USP |
| 7.5 | 75.0 | Glycerol, USP |
| 0.1 | 1.0 | Edetate disodium, USP |
| 0.50 | 5.0 | Boric Acid, NF |
| 0.01 | 0.1 | Benzalkonium chloride, NF |

*Osmolality: 937 mOsm/kg

For each of the above formulations, pH was adjusted to 6.0 using either sodium hydroxide, 0.5N or hydrochloric acid, 0.5N, and each formulation was QS to 1 mL using purified water (USP).

The study was conducted in a hotel setting, similar to the study described in Example 11. Subjects were screened during the evening and lid swelling was subjectively assessed. Morning baseline lid swelling was assessed immediately upon awakening using the global lid swelling scale previously described in Example 11. Immediately after morning baseline measurements were assessed, drug was instilled into the eyes of each subject as follows. Eleven subjects received one drop of naphazoline 0.09%/NaCl ophthalmic solution in one eye, and one drop of oxymetazoline 0.05%/NaCl solution in the fellow eye. Another eleven subjects received one drop of naphazoline 0.09%/glycerol 7.5% in one eye, and one drop of oxymetazoline 0.05%/glycerol 7.5% in the fellow eye. Lid swelling was assessed using the global lid swelling scale in 20 minute intervals for the first hour post-treatment, then assessed in 30 minute intervals during the second hour post-treatment, followed by assessment in 1 hour intervals up to 6 hours post-treatment.

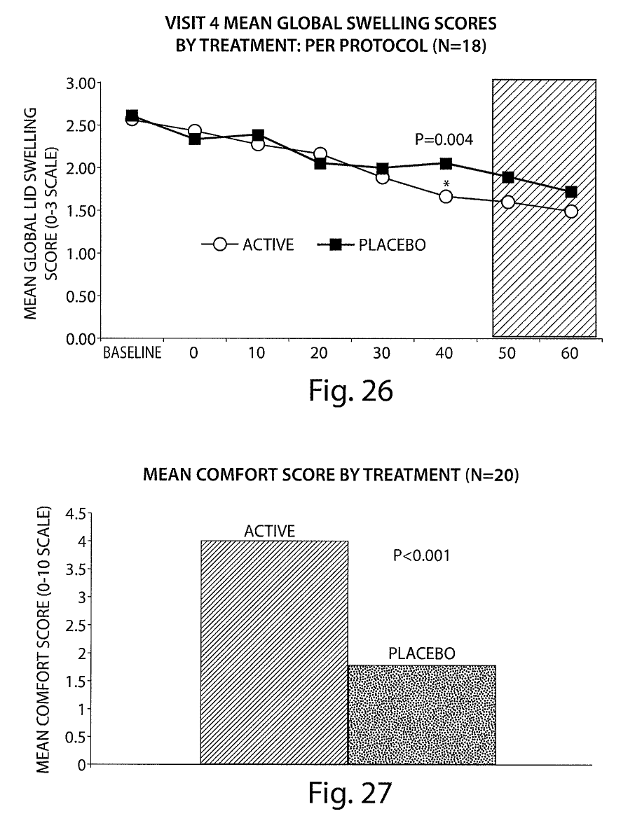

The results are shown in FIGS. 28-31. As shown in FIG. 28, both naphazoline 0.09%/NaCl 3% and oxymetazoline 0.05%/NaCl 3% were effective at reducing morning eyelid swelling over a 6 hour interval, post-treatment. Oxymetazoline 0.05%/NaCl 3% had slightly better reduction in global lid swelling reduction. However, when asked to choose between naphazoline 0.09%/NaCl 3% and oxymetazoline 0.05%/NaCl 3%, 66.6% of the subjects that received both treatments indicated they preferred the naphazoline 0.09%/NaCl 3% ophthalmic solution.

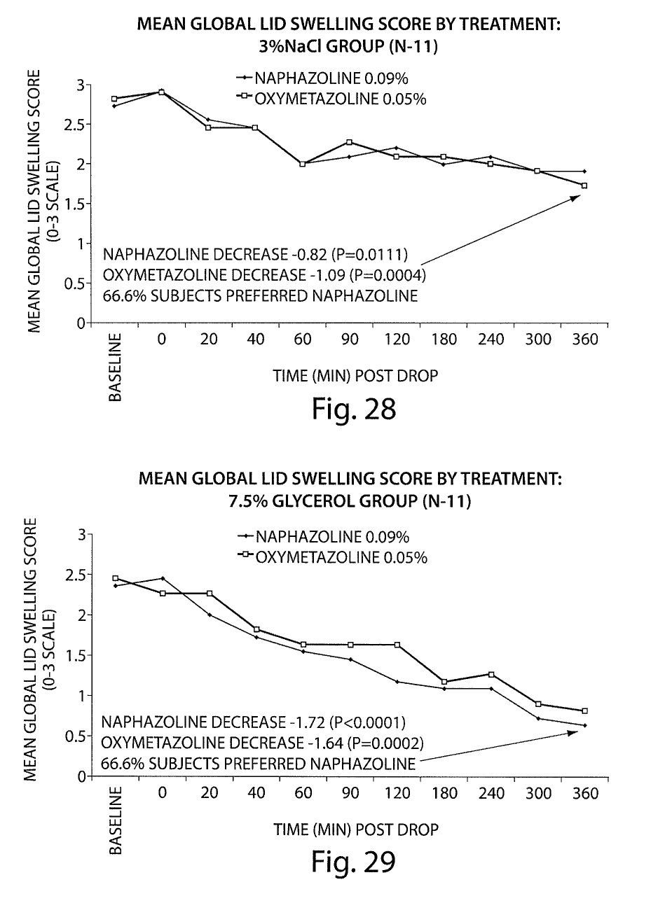

As shown in FIG. 29, both naphazoline 0.09%/glycerol 75% and oxymetazoline 0.05%/glycerol 7.5% were effective at reducing morning eyelid swelling over a 6 hour interval, post-treatment, with naphazoline 0.09%/glycerol 7.5% yielding slightly better reduction. When asked to choose between the two ophthalmic solutions, 66.6% of the subjects indicated they preferred the naphazoline 0.09%/glycerol 7.5% solution.

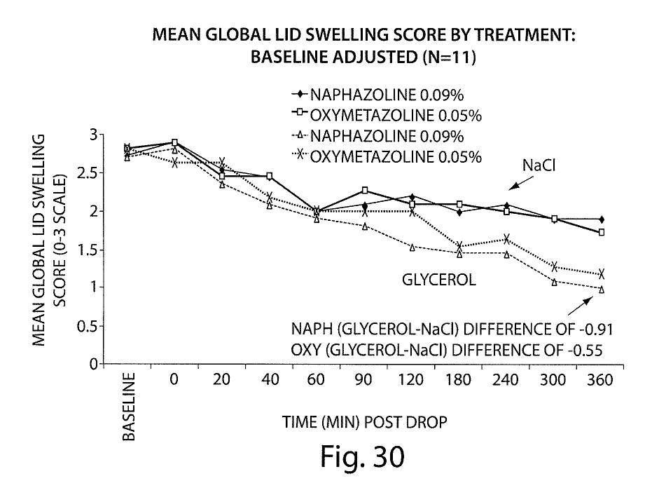

FIG. 30 shows a comparison of the efficacy of all 4 ophthalmic solutions tested, at reducing morning eyelid swelling (for comparison, the different treatment groups were normalized to the same baseline (i.e., pre-drop instillation) value. As shown in FIG. 30, the glycerol 7.5% based solutions (i.e., naphazoline 0.09%/glycerol 7.5% and oxymetazoline 0.05%/glycerol 7.5%) were more effective than the NaCl 3% based solutions (i.e., naphazoline 0.09%/NaCl 3% and oxymetazoline 0.05%/NaCl 3%), which was surprising and unexpected. One of skill in the art would expect that NaCl, a higher tonicity agent than glycerol, would be more effective at reducing morning lid swelling. However, the results indicate that the glycerol based-solution were more effective.

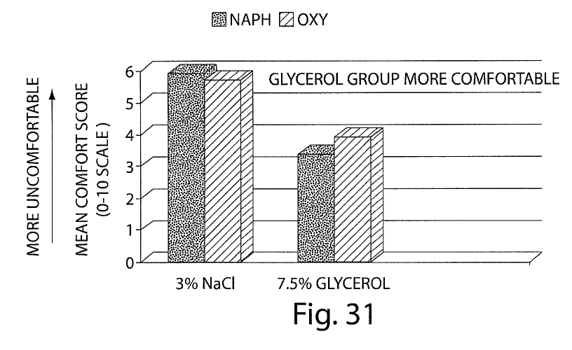

Subjects were also asked to grade comfort of the drop in their eye on a subjective scale of 0-10 (0 indicating most comfortable, 10 indicating least comfortable). The results are shown in FIG. 31. As shown in FIG. 31, the glycerol 7.5% based ophthalmic solutions were found to be more comfortable than the NaCl 3% based ophthalmic solutions, although each of the glycerol 7.5% based and NaCl 3% based solutions were within the targeted osmolality range for an acceptable comfort profile. Five subjects reported a stinging/burning sensation upon instillation of the NaCl based solutions.

In summary, the glycerol 7.5% based ophthalmic solutions (i.e., naphazoline 0.09%/glycerol 7.5% and oxymetazoline 0.05%/glycerol 7.5%) were more effective at reducing morning eyelid swelling, having a greater and quicker decrease from baseline lid swelling measurements. Additionally, the glycerol based solutions were found to be more comfortable, with no adverse effects reported. Of the formulations tested, although the naphazoline 0.09%/glycerol 7.5% was numerically slightly more effective at reducing global morning lid swelling, the differences were not significant. The naphazoline 0.09%/glycerol 7.5% solution was also found to be more comfortable, and preferred by subjects over the oxymetazoline 0.05%/glycerol 7.5% solution.

### Example 13

### Oxymetazoline 0.05%/Glycerol 7.5% Ophthalmic Solution for the Treatment of Morning Eyelid Edema

A single center, contralateral, study is designed to assess and compare the efficacy of a single dose of oxymetazoline 0.05%/glycerol 7.5% ophthalmic solution in the reduction of morning eyelid swelling in a hotel setting.

4 visits are conducted over approximately 1 week. During visit 1, evening baseline lid swelling is assessed using the subjective regional/global lid swelling scale and scoring system and digital photos, as described in Example 11. Additionally, investigator evaluated ocular redness grading is completed at hourly intervals between 6 pm and 9 pm.

During visit 2, morning baseline lid swelling is assessed using the subjective regional/global lid swelling scale and scoring system, and digital photography. Investigator evaluated ocular redness grading is also assessed immediately following awakening at 20, 40, and 60 minutes, then hourly for 6 hours following awakening.

During visit 3, evening lid swelling and ocular redness is assessed using the same procedures as for visit 1.

During visit 4, morning lid swelling is assessed according to the procedures used for visit 2. Patients are then randomized to one of the treatment arms and receive 1 drop of study medication in both eyes. Following instillation, the same study assessments and times are completed as at visit 2. Comfort of the formulations is also subjectively assessed, as previously described (0-10 scale, 0 being more comfortable 10 being less comfortable).

The treatment arms are as follows:

Formulation 1: Oxymetazoline 0.05% ophthalmic solution
Formulation 2: Oxymetazoline 0.05%/Glycerol 7.5% ophthalmic solution
Formulation 3: Vehicle of Formulation 1 (no oxymetazoline, no glycerol)

### Formulation 4: Vehicle of Formulation 2 (Glycerol 7.5%, no Oxymetazoline)

3D scanning technology is also used to assess and compare the efficacy of these four formulations in the reduction of morning eyelid swelling. Baseline scans are performed per subject and eye using a 3D scanner in the evening and following morning, prior to treatment. Patients are then randomized to one of the treatment arms and receive 1 drop of study medication in both eyes. Immediately following instillation, 3D scans of each eye are taken in regular time intervals.

### Example 14

### New Combined Osmotic Agent/Vasoconstrictor Formulations for the Treatment of Morning Eyelid Edema

Approximately 0.5 grams of the following chemicals were added to separate 50 mL polypropylene tubes. The process was done in duplicate.

### 1. Caffeine

### 2. Carbomer 934P

### 3. Tannic Acid

### 4. Ascorbic Acid

### 5. Dextran 40,000

### 6. Inulin

### 7. Mannitol

8. Menthol (0.05 grams)
9. Menthol (0.05 grams), Polysorbate 80 (0.50 grams)

To each of tubes 1-9, 50 mL Oxymetazoline Hydrochloride sample was added and vortexed. To the remaining 9 tubes, 50 mL naphazoline hydrochloride sample was added and vortexed. Each solution was tested for pH and osmolality. The results are shown in Table 6 below.

TABLE 6
pH and Osmolality of Various Lid Swelling Formulations

| Sample | pH | Osmolality (mOsm/kg) |
|---|---|---|
| BCL393-028-1A | 5.99 | 1035 |
| Naphazoline/Caffeine | | |
| BCL393-028-1B | 6.08 | |
| Naphazoline/Carbomer 934P * | | |
| BCL393-028-1C | 5.98 | 1038 |
| Naphazoline/Tannic Acid | | |
| BCL393-028-1D | 6.02 | 1098 |
| Naphazoline/Ascorbic Acid | | |
| BCL393-028-1E | 5.94 | 1005 |
| Naphazoline/Dextran 40,000 | | |
| BCL393-028-1F | 5.90 | 1011 |
| Naphazoline/Inulin | | |
| BCL393-028-1G | 5.97 | 1095 |
| Naphazoline/Mannitol | | |
| BCL393-028-1H | 5.98 | 1011 |
| Naphazoline/Menthol | | |
| BCL393-028-1I | 6.00 | 1005 |
| Naphazoline/Menthol, | | |
| Polysorbate 80 | | |
| BCL393-028-2A | 6.07 | 948 |
| Oxymetazoline/Caffeine | | |
| BCL393-028-2B | 6.06 | |
| Oxymetazoline/Carbomer 934P * | | |
| BCL393-028-2C | 5.97 | 954 |
| Oxymetazoline/Tannic Acid | | |
| BCL393-028-2D | 6.00 | 1029 |
| Oxymetazoline/Ascorbic Acid | | |
| BCL393-028-2E | 6.03 | 927 |
| Oxymetazoline/Dextran 40,000 | | |
| BCL393-028-2F | 6.03 | 957 |
| Oxymetazoline/Inulin | | |
| BCL393-028-2G | 5.95 | 1023 |
| Oxymetazoline/Mannitol | | |
| BCL393-028-2H | 5.97 | 1035 |
| Oxymetazoline/Menthol | | |
| BCL393-028-2I | 5.98 | 1035 |
| Oxymetazoline/Menthol, | | |
| Polysorbate 80 | | |

* Note:
Upon attempting pH adjustment, solution became gel form.

Each of the above formulations are tested for efficacy at reducing morning lid swelling, using 3D scanning technology, as described in Examples 1-9 above, and using the subjective regional/global lid swelling scale and scoring system, as described above in Examples 11 and 12. Comfort of the formulation is subjectively assessed, as previously described (scale 0-10, 0 being more comfortable, 10 being less comfortable).

### EQUIVALENTS

The present invention provides in part topical ophthalmic formulations for use in treating eyelid swelling. While specific embodiments of the subject invention have been discussed, the above specification is illustrative and not restrictive. Many variations of the invention will become apparent to those skilled in the art upon review of this specification. The appendant claims are not intended to claim all such embodiments and variations, and the full scope of the invention should be determined by reference to the claims, along with their full scope of equivalents, and the specification, along with such variations.

All publications and patents mentioned herein, including those items listed below, are hereby incorporated by reference in their entireties as if each individual publication or patent was specifically and individually indicated to be incorporated by reference. In case of conflict, the present application, including any definitions herein, will control.

### REFERENCES

Juniper E F, Guyatt G H, and Dolovich J. 1994. Assessment of quality of life in adolescents with allergic rhinoconjunctivitis: Development and testing of a questionnaire for clinical trials. JAllergy Clin Immunol. 93: 413-423.
Beltrani V S. 2001. Eyelid dermatitis. Curr Allergy Asthma Rep. 1: 380-388. [0113] Zide B M and Jelks G W. 1985. The eyelids. Chapter 3, pp. 21-32, in Surgical Anatomy of the Orbit. Raven, New York.
Wobig J. 1982. Eyelid anatomy. Chapter 7, pp. 78-87, in Cosmetic Oculoplastic Surgery, Putterman A M, ed. Grune & Stratton, New York.
Langley K E, Patrinely J R, Anderson R L, and Thiese S M. 1987. Unilateral blepharochalasis. Ophthalmic Surg. 18: 594-598.
American Society for Aesthetic Plastic Surgery (ASAPS). News release: Mar. 4, 2003. Available at: http://www.surgery.org/news_releases/mar0303stats.html.
Kolker A E. 1970. Hyperosmotic solutions in glaucoma. Investigative Opthalmology. 9: 418-423.
Bielory L. 2000. Allergic and immunologic disorders of the eye. Part II: Ocular allergy. JAllergy Clin Immunol. 106: 1019-1032.
Carter B B. 1999. Eye swelling and pain: a Chinese herbal case study. www.pulsemed.org.
Greiner J V, Peace D G, Baird R S, and Allansmith M R. 1985. Effects of eye rubbing on the conjunctiva as a model of ocular inflammation. Am J. Opthalmol. 100: 45-50.
Chen D M and Crosby D L. 1997. Periorbital edema as an initial presentation of rosacea. J Am Acad Dermatol. 37: 346-348.
Smith N H, Rados W T, Cohen F B, and Cinotti A A. 1977. Malignant lymphoma presenting as bilateral swelling of the eyelid. J Med Soc NJ. 74: 968-970. [0123] Jacobson D M. 2000. Dysthyroid orbitopathy. Semin Neurol. 20: 43-54.
Dupouy-Camet J, Kociecka W, Bruschi F, et al. 2002. Opinion on the diagnosis and treatment of human trichinellosis. Expert Opin Pharmacother. 3: 1117-1130.

We claim:

1. A topical pharmaceutical composition for the treatment and prevention of eyelid swelling in a subject comprising a pharmaceutically acceptable carrier and an effective amount of an osmotically active agent and a vasoconstrictor, wherein the osmotically active agent is NaCl 3% w/v or glycerol 7.5% w/v, and vasoconstrictor is naphazoline 0.09% w/v or oxymetazoline 0.05% w/v.

2. The composition of claim 1, wherein the osmotic agent is glycerol 7.5% w/v and the vasoconstrictor is naphazoline 0.09% w/v.

3. The composition of claim 1, wherein the osmotic agent is glycerol 7.5% w/v and the vasoconstrictor is oxymetazoline 0.05% w/v.

4. The composition of claim 1, wherein the osmotic agent is NaCl 3% w/v and the vasoconstrictor is naphazoline 0.09% w/v.

5. The composition of claim 1, wherein the osmotic agent is NaCl 3% w/v and the vasoconstrictor is oxymetazoline 0.05% w/v.

6. The composition of claim 1, wherein the osmolality of the composition is between 500 and 1050 mOsm/Kg.

7. A topical pharmaceutical composition for the treatment and prevention of eyelid swelling in a subject comprising a pharmaceutically acceptable carrier and 0.9 mg/mL naphazoline hydrochloride, 30 mg/mL sodium chloride, 1 mg/mL edetate disodium, 5 mg/mL boric acid, and 0.1 mg/mL benzalkonium chloride, wherein the pH of the composition is 6.0.

8. A topical pharmaceutical composition for the treatment and prevention of eyelid swelling in a subject comprising a pharmaceutically acceptable carrier and 0.9 mg/mL naphazoline hydrochloride, 75 mg/mL glycerol, 1 mg/mL edetate disodium, 5 mg/mL boric acid, and 0.1 mg/mL benzalkonium chloride, wherein the pH of the composition is 6.0.

9. A topical pharmaceutical composition for the treatment and prevention of eyelid swelling in a subject comprising a pharmaceutically acceptable carrier and 0.5 mg/mL oxymetzoline hydrochloride, 30 mg/mL sodium chloride, 1 mg/mL edetate disodium, 5 mg/mL boric acid, and 0.1 mg/mL benzalkonium chloride, wherein the pH of the composition is 6.0.

10. A topical pharmaceutical composition for the treatment and prevention of eyelid swelling in a subject comprising a pharmaceutically acceptable carrier and 0.5 mg/mL oxymetazoline hydrochloride, 75 mg/mL glycerol, 1 mg/mL edetate disodium, 5 mg/mL boric acid and 0.1 mg/mL benzalkonium chloride, wherein the pH of the composition is 6.0.

11. A topical pharmaceutical composition for the treatment and prevention of eyelid swelling in a subject comprising a pharmaceutically acceptable carrier and an effective amount of an osmotically active agent and a vasoconstrictor, wherein the osmotically active agent is selected from the group consisting of caffeine, carbomer 934P, tannic acid, ascorbic acid, dextran 40,000, inulin, mannitol, menthol, and polysorbate 80, and wherein the vasoconstrictor is selected from the group consisting of naphazoline, oxymetazoline, phenylephrine, and tetrahydrozoline.

12. A method of treating and preventing eyelid swelling in a subject comprising: administering to the eye surface of the subject a composition comprising an effective amount of an osmotically active agent and a vasoconstrictor, wherein the osmotically active agent is NaCl 3% w/v or glycerol 7.5% w/v, and vasoconstrictor is naphazoline 0.09% w/v or oxymetazoline 0.05% w/v.

13. The method of claim 12, wherein the osmotic agent is glycerol 7.5% w/v and the vasoconstrictor is naphazoline 0.09% w/v.

14. The method of claim 12, wherein the osmotic agent is glycerol 7.5% w/v and the vasoconstrictor is oxymetazoline 0.05% w/v.

15. The method of claim 12, wherein the osmotic agent is NaCl 3% w/v and the vasoconstrictor is naphazoline 0.09% w/v.

16. The method of claim 12, wherein the osmotic agent is NaCl 3% w/v and the vasoconstrictor is oxymetazoline 0.05% w/v.

17. A method of treating and preventing eyelid swelling in a subject comprising: administering to the eye surface of the subject a composition comprising an effective amount of an osmotically active agent and a vasoconstrictor, wherein the osmotically active agent is selected from the group consisting of caffeine, carbomer 934P, tannic acid, ascorbic acid, dextran 40,000, inulin, mannitol, menthol, and polysorbate 80, and wherein the vasoconstrictor is selected from the group consisting of naphazoline, oxymetazoline, phenylephrine, and tetrahydrozoline.

18. A kit comprising a pharmaceutical composition of any of claims 1 or 11.
